(19) United States
(12) Patent Application Publication
(10) Pub. No.: US 2020/0268821 A1
(43) Pub. Date: Aug. 27, 2020

(54) HERBAL COMPOSITIONS WITH IMPROVED BIOAVAILABILITY

(71) Applicant: Receptor Holdings, Inc., Seattle, WA (US)

(72) Inventors: Andrea Leone-Bay, Ridgefield, CT (US); Gregory Wesner, Bainbridge Island, WA (US)

(73) Assignee: Receptor Holdings, Inc., Seattle, WA (US)

(21) Appl. No.: 16/753,721

(22) PCT Filed: Oct. 5, 2018
(86) PCT No.: PCT/US18/54728

§ 371 Date: Apr. 3, 2020

Related U.S. Application Data
(60) Provisional application No. 62/568,678, filed on Oct. 5, 2017.

(51) Int. Cl. A61K 36/752; A61K 31/20; A61K 33/06; A61K 33/18; A61K 33/26; A61K 33/30; A61K 31/07; A61K 31/122; A61K 31/355; A61K 31/375; A61K 31/4415; A61K 31/51; A61K 31/525; A61K 31/714; A61K 31/59; A61K 36/9066; A61K 36/81; A61K 36/53; A61K 36/61; A61K 36/36; A61K 36/51; A23L 33/105; A23L 33/17; A23L 33/15

(52) U.S. Cl. CPC A61K 36/752; A61K 9/0056; A61K 33/06; A61K 33/18; A61K 33/26; A61K 33/30; A61K 31/07; A61K 31/122; A61K 31/355; A61K 31/375; A61K 31/4415; A61K 31/51; A61K 31/525; A61K 31/714; A61K 31/59; A61K 36/9066; A61K 36/81; A61K 36/53; A61K 36/61; A61K 36/36; A61K 36/51; A23L 33/105; A23L 33/17; A23L 33/15; A61K 31/20

(57) ABSTRACT

Herbal compositions in various carrier combinations are described. The carriers can include N-acylated fatty amino acids, penetration enhancers, and/or various other beneficial carriers. The herbal composition/carrier combinations can create administration benefits.

### CROSS-REFERENCE TO RELATED APPLICATION

This application claims priority to 62/568,678 filed on Oct. 5, 2017, which is incorporated herein by reference in its entirety as if fully set forth herein.

### FIELD OF THE DISCLOSURE

The current disclosure provides herbal compositions with improved bioavailability in various carrier combinations. The carriers can include N-acylated fatty amino acids, penetration enhancers, and/or various other beneficial carriers. The herbal composition/carrier combinations can create administration benefits following oral administration.

### BACKGROUND OF THE DISCLOSURE

Historically, the plant world has been the most important source of medicinal agents for the treatment of human and animal disease, and for use as preventative agents in maintaining good health. However, for at least the last 150 years, Western medicine has been dominated by synthetic chemical agents.

It is now being increasingly recognized, however, that many plant-based compositions are highly effective agents for the prevention and treatment of disease. A single plant can possess a large number of pharmaceutically active agents, and extracts obtained therefrom can exert their activities on a variety of physiologic processes, increasing the range of the desired therapeutic effect. In general, the pharmaceutically active agents found in plants fall into four major classes: alkaloids, glycosides, polyphenols and terpenes.

As one example, U.S. Publication No. 2015/0050373 describes use of plants from the Calophyllum genus to treat metabolic disorders. Calophyllum is a flowering plant genus of 180-200 species of tropical evergreen trees. The Calophyllum genus includes four subcategories: Calophyllum brasiliense, Calophyllum caledonicurn, Calophyllum inophyllum and Calophyllum soulattri. Calophyllum inophyllum, is a medium to large sized evergreen tree that averages 25-65 feet in height. Different medicinal uses of this plant have been reported in the literature, for example, decoction of the bark of this plant treats internal hemorrhages. The oil extracted from Calophyllum inophyllum seeds is used to treat rheumatoid arthritis or joint disorders; itching; eczema; pimples appearing on the head; eye diseases; and kidney failure.

U.S. Publication No. 2014/0193345 describes use of the plants Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Camellia sinensis and Krameria triandra to treat mucosal lesions.

U.S. Publication No. 2010/0068297 describes use of the plants Punica granatum, Viburnum plicatum, Camellia sinensis, and Acer spp. as antimicrobials.

U.S. Pat. No. 5,401,777 describes use of the plant Curcuma longa to treat chronic inflammatory bowel disease, chronic hepatitis, chronic bronchial asthma, and psoriasis.

Butterweck, V. CNS Drugs. 2003; 17(8):539-62 reviews research demonstrating the antidepressant effects of the plant Hypericum perforatum, also known as St. John's wort. Hypericum perforatum may also be useful for treating menopause, inflammation, infection, pain, anxiety, and insomnia.

U.S. Publication No. 2008/0254135 describes use of Polygonum cuspidatum and grape skin extracts as dietary supplements to provide antioxidants and promote general health and well-being. Antioxidants may help prevent cancer by preventing free radical-induced DNA damage.

While plants and plant extracts have been used to promote health throughout human history, researchers are now beginning to identify some of the active components that are responsible for the health benefits of certain plants. For example, curcumin is an active component of Curcuma longa, and has been shown to have anti-inflammatory and anti-cancer properties. As another example, hypericin is an active component of St. John's wort, or Hypericum perforatum. As another example, resveratrol is an active component present in Polygonum cuspidatum (i.e., Japanese knotweed) and the skins of many fruits such as grapes, blueberries, raspberries, and mulberries.

Despite the numerous health benefits associated with many plants and plant extracts, their active components can have low bioavailability when orally ingested, detracting from their usefulness in some instances. For example, curcumin, hypericin, and resveratrol are plant active components with low oral bioavailability. Thus, as these examples provide, there is room for improvement in the oral administration of herbal compositions.

### SUMMARY OF THE DISCLOSURE

The current disclosure provides herbal compositions with improved bioavailability formulated for oral delivery. By providing improved bioavailability, physiological benefits are enhanced, increasing the usefulness of these compounds.

The disclosed herbal compositions can create various administration benefits in providing therapeutically effective amounts in a variety of conditions. Exemplary administration benefits include increased absorption, increased bioavailability, faster onset of action, higher peak concentrations, faster time to peak concentrations, increased subjective therapeutic efficacy, and increased objective therapeutic efficacy.

The improved bioavailability of the herbal compositions is created by including one or more N-acylated fatty amino acids, absorption enhancing agents, and/or various other beneficial carriers, such as surfactants, detergents, azones, pyrrolidones, glycols and bile salts in an oral formulation. In particular embodiments, N-acylated fatty amino acids can be linear, branched, cyclic, bicyclic, or aromatic including, for example, 1-50 carbon atoms in an oral formulation. That use of N-acylated fatty amino acids could provide a bioavailability benefit with an herbal composition was unexpected given particular aspects of herbal compositions described further herein. For example, the ability of N-acylated fatty amino acids to increase absorption of compounds is proportional to the water-solubility of a compound. Many herbal compounds present in herbal compositions are not water-soluble and would not have been expected to be affected by the presence of an N-acylated fatty amino acid.

In particular embodiments, the herbal compositions include Calophyllum brasiliense, Calophyllum caledonicurn, Calophyllum inophyllum, Calophyllum soulattri, Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Camellia sinensis, Krameria triandra, Punica granatum, Viburnum plicatum, Nicotiana tabacum, Duboisia hopwoodii, Asclepias syriaca, Curcuma longa, Hypericum perforatum, Polygonum cuspidatum, Vitis spp., Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., Mentha spp., Cannabis sativa, Cannabis indica, Cannabis ruderalis, and/or Acer spp., or an extract and/or an active component thereof.

In particular embodiments, the herbal compositions include an active component of a plant, such as capsaicin, reserpine, vinblastine, hesperidin, naringin, rutin, quercitrin, curcumin, hypericin, resveratrol, catechin eugenol, limonene, or linalool.

### BRIEF DESCRIPTION OF THE FIGURES

FIGS. 1A and 1B show an established correlation between water-solubility and the ability of N-[8-(2-hydroxybenzoyl) amino] caprylate (SNAC) to improve a molecule's absorption. FIG. 1A shows the multiple of improvement from SNAC plotted for cromolyn, vitamin B12, atorvastatin, and ibandronate, along with the aqueous solubility of each molecule. The plotted data shows a striking fit to a logarithmic trendline (R²=0.998), indicating a logarithmic relationship between the aqueous solubility of each molecule and the extent to which SNAC improves absorption. As water solubility of the molecule increases, the ability of SNAC to improve its bioavailability also increases. FIG. 1B plots the aqueous solubility of heparin, acyclovir, rhGH, PTH, MT-II, GLP-1, calcitonin, yy peptide, THC, and resveratrol, according to the logarithmic trendline derived from FIG. 1A.

FIG. 2 provides active components of herbal compositions.

FIG. 3 provides modified amino acids of compounds I-XXXV.

FIG. 4 provides fatty acid amino acids of formula (a), (b), (c), (d), (e), (f), (g), (h), (i), (j), (k), (l), (m), (n), (o), (p), (q), and (r), wherein R1 is an alkyl group including 5 to 19 carbon atoms, R2 is H (i.e. hydrogen) or CH3 (i.e. methyl group), and R3 is H; or a salt or the free acid form thereof.

FIGS. 5A and 5B provide the average results of the study comparing onset and duration of action of orally administered cannabis/N-[8-(2-hydroxybenzoyl) amino] caprylate (SNAC, “test”) formulation and cannabis (without SNAC, “control”) formulation.

FIGS. 6A-6F provide the results for each individual participant in the study comparing onset and duration of action of orally administered cannabis/N-[8-(2-hydroxybenzoyl) amino] caprylate (SNAC, “test”) formulation and cannabis (without SNAC, “control”) formulation.

FIG. 7 shows a comparison of intensity, duration and onset of action of orally administered cannabis formulations with a high SNAC dose (200 mg, “high dose”), a low SNAC dose (100 mg, “low dose”) and no SNAC (“control”).

FIG. 8 shows intensity, duration and onset of action of cannabis formulated with SNAC administered orally (“PO”) compared to cannabis administered by inhalation (“INH”).

FIG. 9 shows the values for THC and CBD Cₘₐₓ (ng/ml) and AUC (hr*ng/mL) following a single oral administration to rats

FIG. 10 shows a graph of THC and CBD Cₘₐₓ (ng/ml) and AUC (hr*ng/mL) following a single oral administration to rats.

FIG. 11 shows intensity, duration and onset of action of orally administered cannabis/N-[8-(2-hydroxybenzoyl) amino] caprylic acid (NAC, “test”) formulation and cannabis (without NAC, “control”) formulation.

### DETAILED DESCRIPTION

Despite the numerous health benefits associated with many plants and plant extracts, their active components can have low bioavailability when orally ingested, detracting from their usefulness in some instances. For example, curcumin, hypericin, and resveratrol are plant active components with low oral bioavailability. Thus, as these examples provide, there is room for improvement in the oral administration of herbal compositions. The current disclosure provides herbal extract compositions with improved bioavailability. By providing improved bioavailability the usefulness of these herbal compositions can be increased.

The disclosed herbal compositions with improved bioavailability can create various administration benefits in providing therapeutically effective amounts in a variety of conditions. Exemplary administration benefits include increased absorption, increased bioavailability, faster onset of action, higher peak concentrations, faster time to peak concentrations, increased subjective therapeutic efficacy, and increased objective therapeutic efficacy.

The improved bioavailability of the herbal compositions is created by including one or more N-acylated fatty amino acids, absorption enhancing agents, and/or various other beneficial carriers, such as surfactants, detergents, azones, pyrrolidones, glycols and bile salts in an oral formulation. In particular embodiments, N-acylated fatty amino acids can be linear, branched, cyclic, bicyclic, or aromatic including, for example, 1-50 carbon atoms in an oral formulation. That use of N-acylated fatty amino acids could provide a fast-acting benefit with an herbal composition was unexpected given particular aspects of plant-based components described further herein. For example, the ability of N-acylated fatty amino acids to increase absorption of compounds is proportional to the water-solubility of a compound. Many plant-based compounds are not water-soluble and would not have been expected to be affected by the presence of an N-acylated fatty amino acid.

Molecules that have been shown to have improved absorption when co-administered with an N-acylated fatty amino acid (e.g., SNAC) include water-soluble molecules such as cromolyn, vitamin B12), atorvastatin, ibandronate, heparin, acyclovir, recombinant human growth hormone (rhGH), parathyroid hormone 1-34 (PTH 1-34), α-melanotropin (MT-II), GLP-1, calcitonin, and peptide yy.

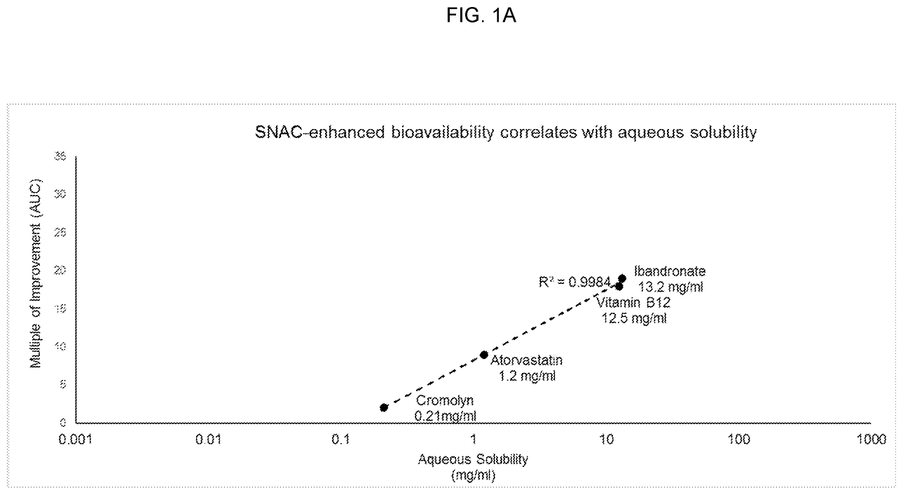

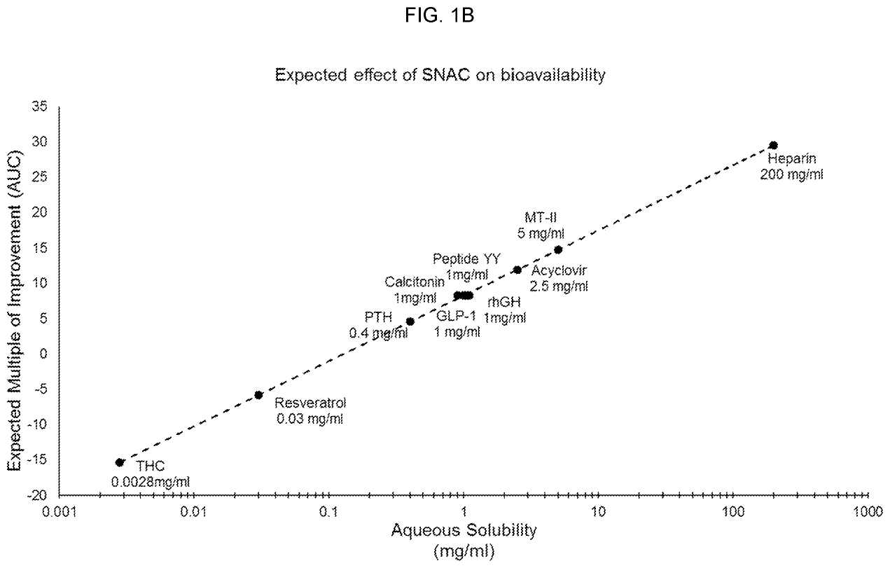

FIG. 1A shows an established correlation between water-solubility and the ability of SNAC to improve a molecule's absorption. For cromolyn, vitamin B12, atorvastatin, and ibandronate, published results include area under the curve (AUC), which is calculated from a time-course of plasma levels. To quantify the effect of co-administration with SNAC, a multiple of improvement can be calculated by dividing the AUC for a molecule with SNAC by the AUC for the molecule without SNAC. FIG. 1A shows the multiple of improvement from SNAC plotted for cromolyn, vitamin B12, atorvastatin, and ibandronate, along with the aqueous solubility of each molecule. The plotted data shows a striking fit to a logarithmic trendline (R²=0.998), indicating a logarithmic relationship between the aqueous solubility of each and the extent to which SNAC improves its absorption.

Heparin, acyclovir, rhGH, PTH, MT-II, GLP-1, calcitonin, and yy peptide are other molecules that have been shown to have SNAC-improved absorption, as demonstrated by Cmax (maximum drug plasma level) and/or Tmax (the time taken to reach maximum drug plasma level). As shown in FIG. 1B, each of these molecules has an aqueous solubility of more than 0.15 mg/ml, and therefore, the model accurately predicts that SNAC can improve their absorption. This result demonstrates that a SNAC-based absorption improvement correlates with a molecule's aqueous solubility. FIG. 1B further plots the aqueous solubility of two plant active components, THC (0.0028 mg/ml) and resveratrol (0.03 mg/ml) to the logarithmic trendline and SNAC's predicted effect based on the same. Based at least on the foregoing, the results described herein were surprising and would not have been reasonably expected by those of ordinary skill in the art.

Aspects of the disclosure are now described in more detail.

The current disclosure provides herbal compositions with improved bioavailability, including vegetable matter and a carrier as an oral formulation. Herbal compositions can include botanical medicines and plant-based nutritional supplements. Herbal compositions, such as botanical medicines, can provide therapeutically-effective amounts to treat a condition, such as those described in the Background of the Disclosure. Nutritional supplements can claim a benefit related to a classical nutrient deficiency; describe how the supplement is intended to affect the structure or function of the human body; characterize a documented mechanism by which the supplement acts to maintain such structure or function; and/or describe general well-being associated with consumption of the product. In particular embodiments, a nutritional supplement may not claim to diagnose, mitigate, treat, cure, or prevent a specific disease or class of diseases.

Herbal compositions include vegetable matter. Vegetable matter is matter produced by a plant and includes any whole plant or plant part (e.g., bark, wood, leaves, stems, roots, flowers, fruits, seeds, or parts thereof) and/or exudates or extracts thereof. In particular embodiments, herbal compositions include botanical products. Botanical products can include plant materials, algae, macroscopic fungi, and/or combinations thereof. In particular embodiments, herbal compositions include a mixture of various types of vegetable matter. Herbal compositions can also include materials derived from vegetable matter including resins, oils (e.g., essential oils), dried flowers, spices, kief, tinctures, infusions, etc. In particular embodiments, the vegetable matter has little or no water solubility. In particular embodiments, herbal compositions do not include synthetic, semi-synthetic, or chemically-modified drugs.

In particular embodiments the herbal compositions include vegetable matter derived from Calophyllum brasiliense, Calophyllum caledonicurn, Calophyllum inophyllum, Calophyllum soulattri, Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Krameria triandra, Punica granatum, Viburnum plicatum, Nicotiana tabacum, Duboisia hopwoodii, Asclepias syriaca, Curcuma longa, Hypericum perforatum, Polygonum cuspidatum, Vitis spp, Camellia sinensis, Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., Mentha spp., Cannabis sativa, Cannabis indica, Cannabis ruderalis and/or Acer spp., or an extract thereof.

In particular embodiments, the herbal compositions include vegetable matter derived from Curcuma longa, Hypericum perforatum, Polygonum cuspidatum, Vitis spp., Camellia sinensis, Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., Mentha spp., or an extract thereof.

In particular embodiments the herbal compositions include an active component of a plant, such as a polyphenol, an alkaloid, a glycoside, or a terpene.

In particular embodiments, the herbal compositions include a polyphenol with low water solubility. Examples of plant-derived polyphenols with low water solubility include curcumin, hypericin, resveratrol, and catechin.

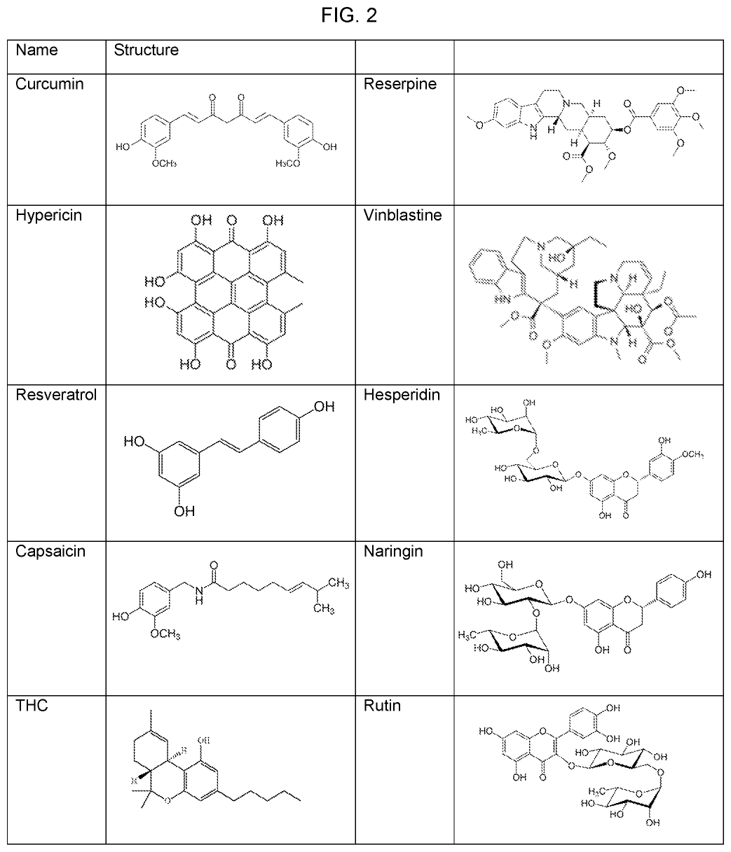

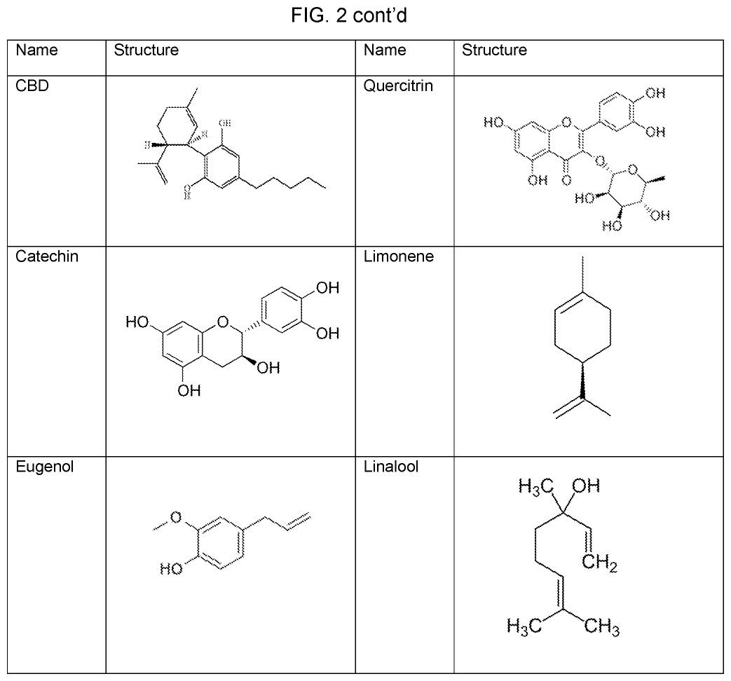

In particular embodiments the herbal compositions include curcumin. Curcumin is a phenolic compound with low water solubility (<0.1 mg/ml) that is responsible for the yellow color of turmeric, a spice derived from Curcuma longa. Curcumin has been shown to have anti-inflammatory and anti-cancer effects, and may be useful for treating chronic inflammatory bowel disease, chronic hepatitis, chronic bronchial asthma, and psoriasis. For the chemical structure of curcumin, see, for example, FIG. 2.

In particular embodiments the herbal compositions include hypericin. Hypericin is a napthodianthrone that is insoluble in water, and is a main active component of Hypericum perforatum, or St. John's wort. Hypericin is used as an anti-depressant, and can also be used for photodynamic cancer therapy because it preferentially accumulates in cancer tissue and causes photosensitivity. For the chemical structure of hypericin, see, for example, FIG. 2.

In particular embodiments the herbal compositions include resveratrol. Resveratrol is a polyphenol with very low water solubility (0.03 mg/ml) and is a main active component of Polygonum cuspidatum, grapes (i.e., plants of the genus Vitis, also referred to as Vitis spp.), blueberries, raspberries, and mulberries. Resveratrol is a potent antioxidant and has anti-inflammatory effects. For the chemical structure of resveratrol, see, for example, FIG. 2.

In particular embodiments the herbal compositions include catechin. Catechin is a polyphenol with low water solubility, and includes four isomers: (−)-epicatechin, (+)-epicatechin, (−)-catechin, and (+)-catechin. Catechin is found in many plants, and dietary sources of catechin include tea (Camellia sinsensis), cocoa (Theobroma cacao), agai, apple, and pear. Catechin is a potent antioxidant and has anti-inflammatory effects. For the chemical structure of catechin, see, for example, FIG. 2.

In particular embodiments the herbal compositions include a plant-derived alkaloid with low water solubility. Examples of plant-derived alkaloids with low solubility include capsaicin, reserpine, and vinblastine.

In particular embodiments the herbal compositions include capsaicin. Capsaicin is an alkaloid with low water solubility that can be used as an analgesic, and for treating neuralgia. Capsaicin is present in the fruits of plants of the genus Capsicum, such as Capsicum annuum, Capsicum chinense, capsicum baccatum, and Capsicum pubescens. For the chemical structure of capsaicin, see, for example, FIG. 2.

In particular embodiments the herbal compositions include reserpine. Reserpine is an indole alkaloid with low water solubility, and is found in the dried roots of plants of the genus Rauwolfia, such as Rauwolfia vomitoria and Rauwolfia serpentina. Reserpine-containing extracts have been used in India for centuries, for treating insanity, fevers, and snakebites. Reserpine is also used to treat hypertension. For the chemical structure of reserpine, see, for example, FIG. 2.

In particular embodiments the herbal compositions include vinblastine. Vinblastine is an alkaloid with low water solubility, and is produced by plants of the genus Vinca, such as Vinca rosea. Vinblastine can block cellular division by disrupting microtubule formation and is used as a chemotherapy agent to treat a variety of cancers. For the chemical structure of vinblastine, see, for example, FIG. 2.

In particular embodiments, the herbal compositions include a glycoside with low water solubility. Examples of plant-derived glycosides with low water solubility include hesperidin, naringin, rutin, and quercitrin.

In particular embodiments the herbal compositions include hesperidin. Hesperidin is a glycoside with low water solubility. Hesperidin is found in the fruit of citrus trees, such as Citrus aurantium, Citrus sinensis, Citrus limon, and Citrus aurantifolia. Hesperidin is an antioxidant, is anti-inflammatory, may help prevent cancer, and is used to treat blood vessel conditions such as hemmorhoids, variscose veins and poor circulation. For the chemical structure of hesperidin, see, for example, FIG. 2.

In particular embodiments the herbal compositions include naringin. Naringin is a glycoside with low water solubility that is present in the fruits of Citrus plants, such as Citrus sinensis, Citrus aurantium, Citrus reticulate, Citrus clementina, and Citrus bergamia. Naringin is an antioxidant, is anti-inflammatory, and can improve glucose regulation. For the chemical structure of naringin, see, for example, FIG. 2.

In particular embodiments the herbal compositions include rutin. Rutin is a glycoside with low water solubility, and is found in buckwheat (Fagopyrum tataricum), Rheum species (such as Rheum rhabarbarum, or rhubarb), and asparagus. Rutin is a potent antioxidant. For the chemical structure of rutin, see, for example, FIG. 2.

In particular embodiments the herbal compositions include quercitrin. Quercitrin is a glycoside with low water solubility, and is formed by the flavonoid quercetin and the deoxy sugar rhamnose. Quercitrin has potent antioxidant properties and is found in a wide variety of plants, such as buckwheat (Fagopyrum tataricum) and St. John's wort (Hypericum perforatum). For the chemical structure of quercitrin, see, for example, FIG. 2.

In particular embodiments the herbal compositions include terpenes. Terpenes are organic molecules that include chains of isoprene subunits, and are typically insoluble in water. Examples of plant-derived terpenes with low water solubility include eugenol, limonene, and linalool.

In particular embodiments the herbal compositions include eugenol. Eugenol is a terpene with low water solubility and has anti-inflammatory effects. Eugenol is found in clove (Syzygium aromaticum) oil, cinnamon, nutmeg, cannabis, and bay leaf. For the chemical structure of eugenol, see, for example, FIG. 2.

In particular embodiments the herbal compositions include limonene. Limonene is a terpene with low water solubility, which forms two isomers. The D-isomer of limonene has a potent orange aroma and can be found in high quantities in Citrus fruits, whereas the L-isomer has a piney aroma and is common in oils extracted from mint (genus Mentha). Limonene is used for weight loss, cancer prevention, to treat bronchitis, and to help control cholesterol levels. For the chemical structure of limonene, see, for example, FIG. 2.

In particular embodiments the herbal compositions include linalool. Linalool is a terpene with low water solubility, which forms two enantiomers known as licareol and coriandrol. Linalool is produced in high quantities by lavender (genus Lavandula), and is produced by many other plants such as birch trees, mint, citrus fruits, and cinnamon. Linalool has sedative, anti-inflammatory, and anxiolytic properties. For the chemical structure of linalool, see, for example, FIG. 2.

Components of herbal compositions can be produced by, e.g., pulverization, decoction, expression, and extraction of a starting plant product. The term “extract” can include all of the many types of preparations containing some or all of the active ingredients found in the relevant plants. Extracts may be produced by cold extraction techniques using a variety of different extraction solvents including water, fatty solvents (such as olive oil), and alcoholic solvents (e.g. 70% ethanol). Cold extraction techniques are typically applied to softer parts of the plant such as leaves and flowers, or in cases wherein the desired active components of the plant are heat labile. Alternatively, the aforementioned solvents may be used to produce extracts of the desired plants by a hot extraction technique, wherein said solvents are heated to a high temperature, the precise value of said temperature being dependent on the properties of the chosen solvent, and maintained at that temperature throughout the extraction process. Hot extraction techniques are more commonly applied to the harder, tougher parts of the plant, such as bark, woody branches and larger roots. In some cases, sequential extractions can be performed in more than one solvent, and at different temperatures. The plant extract may be used in a concentrated form. Alternatively, the extract may be diluted as appropriate to its intended use.

Additional procedures for producing plant extracts (including hot extraction, cold extraction and other techniques) are described in publications including “Medicinal plants: a field guide to the medicinal plants of the Land of Israel (in Hebrew), author: N. Krispil, Har Gilo, Israel, 1986” and “Making plant medicine, author: R. Cech, pub. by Horizon Herbs, 2000”.

In particular embodiments, plant components of herbal compositions (e.g., plant extracts) may be sterilized, for example by autoclaving, and then allowed to cool and stored at an appropriate temperature (e.g., −20° C.). In particular embodiments, further purification to a molecular weight cut-off (e.g., below 10,000 Da) can be carried out, for example, by membrane ultrafiltration before storage.

In particular embodiments, herbal compositions include carriers such as modified amino acids, a surfactant, a detergent, an azone, a pyrrolidone, a glycol, or a bile salt. An amino acid is any carboxylic acid having at least one free amine group and includes naturally occurring, non-naturally occurring and synthetic amino acids. Poly amino acids are either peptides or two or more amino acids linked by a bond formed by other groups which can be linked, e.g. an ester, anhydride, or an anhydride linkage. Peptides are two or more amino acids joined by a peptide bond. Peptides can vary in length from dipeptides with two amino acids to poly peptides with several hundred amino acids. See Chambers Biological Dictionary, editor Peter M. B. Walker, Cambridge, England: Chambers Cambridge, 1989, page 215. Di-peptides, tri-peptides, tetra-peptides, and penta-peptides can also be used.

Carriers which are modified amino acids include acylated fatty acid amino acids (FA-aa) or a salt thereof, which are typically prepared by modifying the amino acid or an ester thereof by acylation or sulfonation. Acylated fatty acid amino acids include N-acylated FA-aa or an amino acid acylated at its alpha amino group with a fatty acid.

Exemplary N-acylated fatty amino acid salts include sodium N-[8-(2-hydroxybenzoyl) amino] caprylate (SNAC). Other names for SNAC include Sodium-N-salicyloyl-8-aminocaprylate, Monosodium 8-(N-salicyloylamino) octanoate, N-(salicyloyl)-8-aminooctanoic acid monosodium salt, monosodium N-{8-(2-hydroxybenzoyl)amino}octanoate, or sodium 8-[(2-hydroxybenzoyl)amino]octanoate. SNAC has the structure:

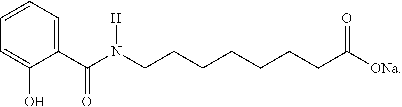

Salts of SNAC may also be used as a carrier.

In particular embodiments, the carriers include:

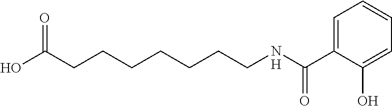

In particular embodiments, the carriers include N-[8-(2-hydroxybenzoyl) amino] caprylic acid (NAC).

Other forms of carriers include:

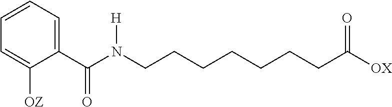

wherein X and Z are independently H, a monovalent cation, a divalent metal cation, or an organic cation. Examples of monovalent cations include sodium and potassium. Examples of divalent cations include calcium and magnesium. Examples of organic cations include ammonium and tetramethylammonium.

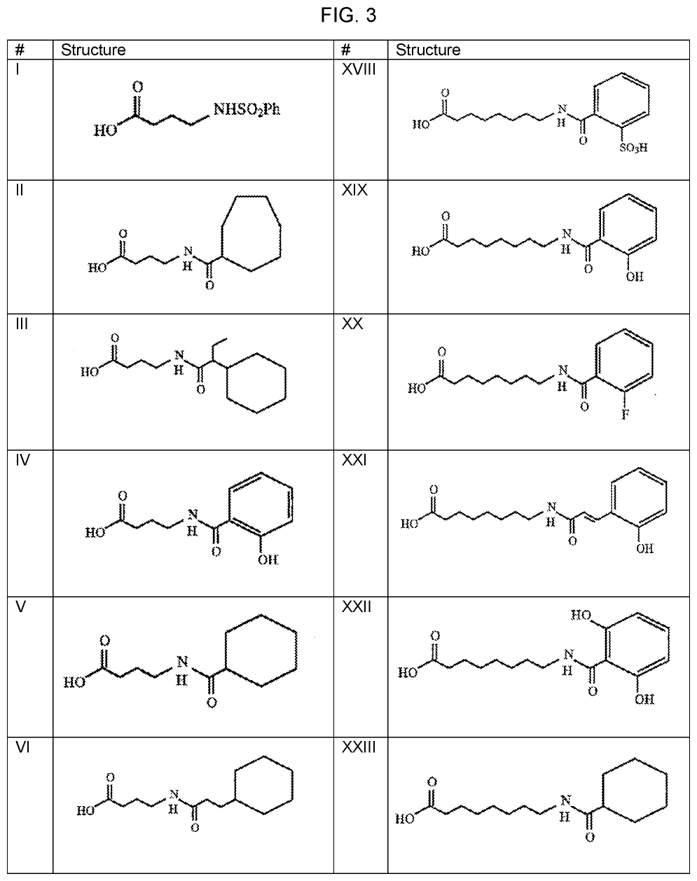

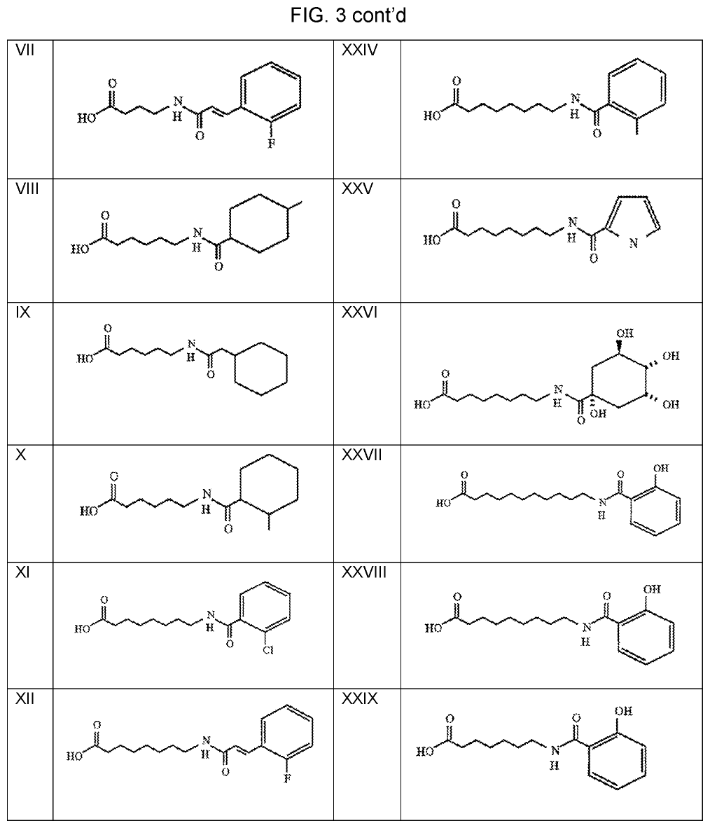

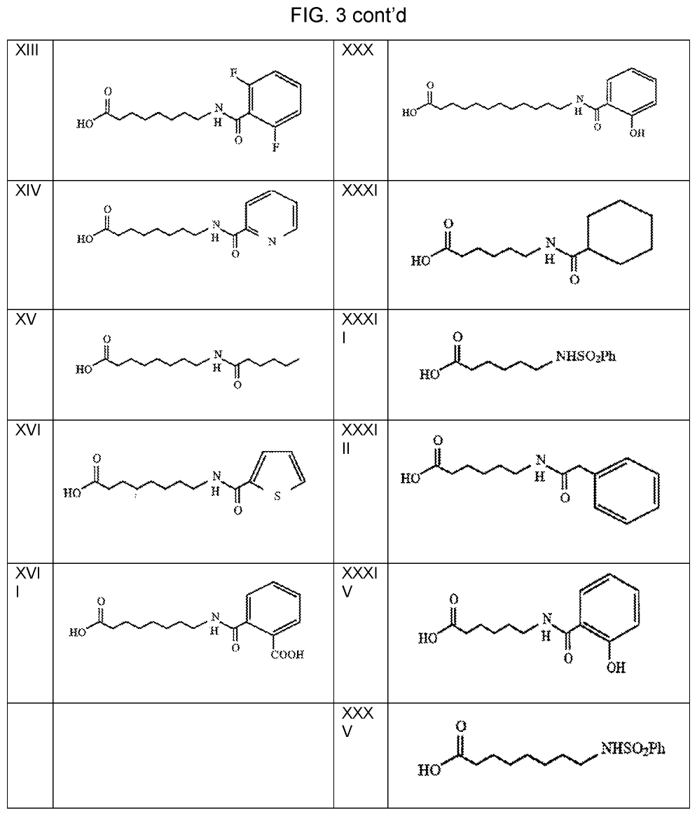

Exemplary modified amino acids, such as N-acylated FA-aas, are provided as compounds I-XXXV (see FIG. 3). Salts of these compounds and other N-acylated FA-aa can also be used as carriers.

Many of the compounds can be readily prepared from amino acids by methods within the skill of those in the art based upon the present disclosure. For example, compounds I-VII are derived from aminobutyric acid. Compounds VIII-X and XXXI-XXIIV are derived from aminocaproic acid. Compounds XI-XXVI and XXXV are derived from aminocaprylic acid. For example, the modified amino acid compounds above may be prepared by reacting the single amino acid with the appropriate modifying agent which reacts with free amino moieties present in the amino acids to form amides. Protecting groups may be used to avoid unwanted side reactions as would be known to those skilled in the art.

The amino acid can be dissolved in aqueous alkaline solution of a metal hydroxide, e.g., sodium or potassium hydroxide, and heated at a temperature ranging between 5° C. and 70° C., in particular embodiments between 10° C. and 40° C., for a period ranging between 1 hour and 4 hours, and in particular embodiments 2.5 hours. The amount of alkali employed per equivalent of NH₂ groups in the amino acid generally ranges between 1.25 and 3 mmole, in particular embodiments between 1.5 and 2.25 mmole per equivalent of NH₂. The pH of the solution generally ranges between 8 and 13, in particular embodiments ranging between 10 and 12.

Thereafter, the appropriate amino acid modifying agent is added to the amino acid solution while stirring. The temperature of the mixture is maintained at a temperature generally ranging between 5° C. and 70° C., in particular embodiments between 10° C. and 40° C., for a period ranging between 1 and 4 hours. The amount of amino acid modifying agent employed in relation to the quantity of amino acid is based on the moles of total free NH₂ in the amino acid. In general, the amino acid modifying agent is employed in an amount ranging between 0.5 and 2.5 mole equivalents, in particular embodiments between 0.75 and 1.25 equivalents, per molar equivalent of total NH₂ group in the amino acid.

The reaction is quenched by adjusting the pH of the mixture with a suitable acid, e.g., concentrated hydrochloric acid, until the pH reaches between 2 and 3. The mixture separates on standing at room temperature to form a transparent upper layer and a white or off-white precipitate. The upper layer is discarded, and the modified amino acid is collected from the lower layer by filtration or decantation. The crude modified amino acid is then dissolved in water at a pH ranging between 9 and 13, in particular embodiments between 11 and 13. Insoluble materials are removed by filtration and the filtrate is dried in vacuo. The yield of modified amino acid generally ranges between 30 and 60%, and usually 45%.

If desired, amino acid esters, such as, for example benzyl, methyl, or ethyl esters of amino acid compounds, may be used to prepare the modified amino acids. The amino acid ester, dissolved in a suitable organic solvent such as dimethylformamide, pyridine, or tetrahydrofuran can be reacted with the appropriate amino acid modifying agent at a temperature ranging between 5° C. and 70° C., in particular embodiments 25° C., for a period ranging between 7 and 24 hours. The amount of amino acid modifying agent used relative to the amino acid ester is the same as described above for amino acids. This reaction may be carried out with or without a base such as, for example, triethylamine or diisopropylethylamine.

Thereafter, the reaction solvent is removed under negative pressure and the ester functionality is removed by hydrolyzing the modified amino acid ester with a suitable alkaline solution, e.g. 1N sodium hydroxide, at a temperature ranging between 50° C. and 80° C., in particular embodiments 70° C., for a period of time sufficient to hydrolyze off the ester group and form the modified amino acid having a free carboxyl group. The hydrolysis mixture is then cooled to room temperature and acidified, e.g. aqueous 25% hydrochloric acid solution, to a pH ranging between 2 and 2.5. The modified amino acid precipitates out of solution and is recovered by conventional means such as filtration or decantation. Benzyl esters may be removed by hydrogenation in an organic solvent using a transition metal catalyst.

The modified amino acid may be purified by recrystallization or by fractionation on solid column supports. Suitable recrystallization solvent systems include acetonitrile, methanol and tetrahydrofuran. Fractionation may be performed on a suitable solid column supports such as alumina, using methanol/n-propanol mixtures as the mobile phase; reverse phase column supports using trifluoroacetic acid/acetonitrile mixtures as the mobile phase; and ion exchange chromatography using water as the mobile phase. When anion exchange chromatography is performed, in particular embodiments a subsequent 0-500 mM sodium chloride gradient is employed.

In particular embodiments, modified amino acids having the formula

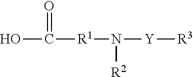

wherein Y is

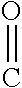

### or SO₂;

R¹ is C₃-C₂₄ alkylene, C₂-C₂₀ alkenylene, C₂-C₂₀ alkynylene, cycloalkylene, or an aromatic, such as arylene;
R² is hydrogen, C₁-C₄ alkyl, or C₂-C₄ alkenyl; and
R³ is C₁-C₇ alkyl, C₃-C₁₀ cycloalkyl, aryl, thienyl, pyrrolo, or pyridyl, and
R³ is optionally substituted by one or more C₁-C₅ alkyl group, C₂-C₄ alkenyl group, F, Cl, OH, OR¹, SO₂, COOH, COOR¹ or, SO₃H;
may be prepared by
reacting in water and the presence of a base a lactam having the formula

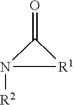

with a compound having the formula R³—Y—X, wherein Y, R¹, R², and R³ are as above and X is a leaving group. A lactam as shown in the above formula can be prepared, for example by the method described in Olah et al., Synthesis, 537-538 (1979).

In particular embodiments, modified amino acids also include an amino acid acylated at its alpha amino group with a fatty acid, which can be represented by the general formula A-X, wherein A is the alpha-amino acid residue and X is a fatty acid attached by acylation to A's alpha-amino group. The amino acids include cationic and non-cationic amino acids. In particular embodiments the term “non-cationic amino acid” refers to an amino acid selected from the group including non-polar hydrophobic amino acids, polar non-charged amino acids, and polar acidic amino acids. In particular embodiments the term “non-cationic amino acid” as used herein refers to amino acids selected from the group including Alanine (Ala), Valine (Val), Leucine (Leu), Isoleucine (Ile), Phenylalanine (Phe), Tryptophan (Trp), Methionine (Met), Proline (Pro), Sarcosine, Glycine (Gly), Serine (Ser), Threonine (Thr), Cysteine (Cys), Tyrosine (Tyr), Asparagine (Asn), Glutamine (Gln), Aspartic acid (Asp), and Glutamic acid (Glu).

In particular embodiments, the acylated FA-aa includes an alpha amino acid residue of a non-polar hydrophobic amino acid. In particular embodiments, the acylated FA-aa may be represented by the general formula A-X, wherein A is the amino acid residue of a non-polar hydrophobic amino acid and X is a fatty acid attached by acylation to A's alpha-amino group. In particular embodiments the term “non-polar hydrophobic amino acid” as used herein refers to categorization of amino acids used by the person ordinarily skilled in the art. In particular embodiments the term “non-polar hydrophobic amino acid” refers to an amino acid selected from the group including Ala, Val, Leu, lie, Phe, Trp, Met, Pro, and Sarcosine.

In particular embodiments, the acylated FA-aa includes the amino acid residue of a polar non-charged amino acid. In particular embodiments the acylated FA-aa may be represented by the general formula A-X, wherein A is the amino acid residue of a polar non-charged amino acid and X is a fatty acid attached by acylation to A's alpha-amino group. In particular embodiments the term “polar non-charged amino acid” as used herein refers to categorization of amino acids used by the person ordinarily skilled in the art. In particular embodiments the term “polar non-charged amino acid” refers to an amino acid selected from the group including Gly, Ser, Thr, Cys, Tyr, Asn, and Gin.

In particular embodiments, the acylated FA-aa includes the amino acid residue of a polar acidic amino acid. In particular embodiments, the acylated FA-aa may be represented by the general formula A-X, wherein A is the amino acid residue of a polar acidic amino acid and X is a fatty acid attached by acylation to A's alpha-amino group. In particular embodiments, the term “polar acidic amino acid” as used herein refers to categorization of amino acids used by the person ordinarily skilled in the art. In particular embodiments, the term “polar acidic amino acid” refers to an amino acid selected from the group including Asp and Glu.

In particular embodiments, the amino acid residue of the acylated FA-aa includes the amino acid residue of an amino acid that is not encoded by the genetic code. Modifications of amino acids by acylation may be readily performed using acylation agents known in the art that react with the free alpha-amino group of the amino acid.

In particular embodiments, the alpha-amino acids or the alpha-amino acid residues herein are in the L-form unless otherwise stated.

In particular embodiments, the amino acid residue is in the free acid form and/or a salt thereof, such as a sodium (Na+) salt thereof.

Exemplary embodiments of acylated FA-aas may be represented by the general Fa-aa formula I:

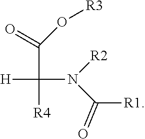

wherein R1 is an alkyl or aryl group including 5 to 19 carbon atoms; R2 is H, CH₃, or covalently attached to R4 via a (CH₂)₃ group; R3 is H or absent; and R4 is an amino acid side chain or covalently attached to R2 via a (CH₂)₃ group; or a salt thereof.

The FA-aa can be acylated with a fatty acid including a substituted or unsubstituted alkyl group including 5 to 19 carbon atoms. In particular embodiments, the alkyl group includes 5 to 17 carbon atoms. In particular embodiments, the alkyl group includes 5-15 carbon atoms. In particular embodiments the alkyl group includes 5-13 carbon atoms. In particular embodiments the alkyl group includes 6 carbon atoms.

In particular embodiments, the acylated FA-aa is soluble at intestinal pH values, particularly in the range pH 5.5 to 8.0, such as in the range pH 6.5 to 7.0. In particular embodiments, the acylated FA-aa is soluble below pH 9.0.

In particular embodiments, the acylated FA-aa has a solubility of at least 5 mg/mL. In particular embodiments, the acylated FA-aa has a solubility of at least 10 mg/mL. In particular embodiments, the acylated FA-aa has a solubility of at least 20 mg/mL. In particular embodiments, the acylated FA-aa has a solubility of at least 30 mg/mL. In particular embodiments, the acylated FA-aa has a solubility of at least 40 mg/mL. In particular embodiments, the acylated FA-aa has a solubility of at least 50 mg/mL. In particular embodiments, the acylated FA-aa has a solubility of at least 60 mg/mL. In particular embodiments, the acylated FA-aa has a solubility of at least 70 mg/mL. In particular embodiments, the acylated FA-aa has a solubility of at least 80 mg/mL. In particular embodiments, the acylated FA-aa has a solubility of at least 90 mg/mL. In particular embodiments, the acylated FA-aa has a solubility of at least 100 mg/mL. In particular embodiments, solubility of the acylated FA-aa is determined in an aqueous solution at a pH value 1 unit above or below pKa of the FA-aa at 37° C. In particular embodiments, solubility of the acylated FA-aa is determined in an aqueous solution at pH 8 at 37° C. In particular embodiments, solubility of the acylated FA-aa is determined in an aqueous solution at a pH value 1 unit above or below pI of the FA-aa at 37° C. In particular embodiments, solubility of the acylated FA-aa is determined in an aqueous solution at a pH value 1 unit above or below pI of the FA-aa at 37° C., wherein said FA-aa includes two or more ionizable groups with opposite charges. In particular embodiments, solubility of the FA-aa is determined in an aqueous 50 mM sodium phosphate buffer, pH 8.0 at 37° C.

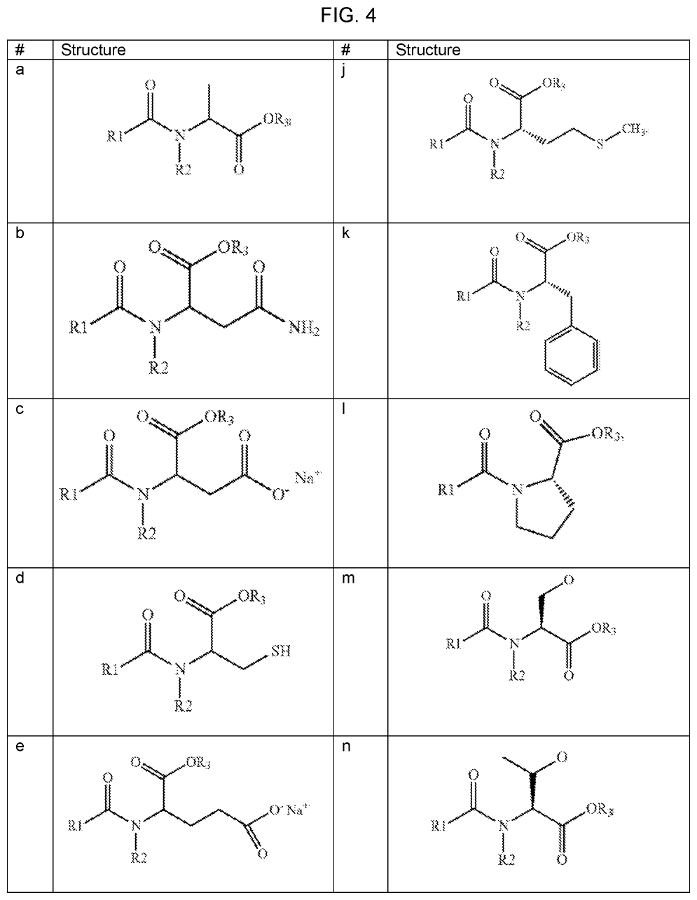

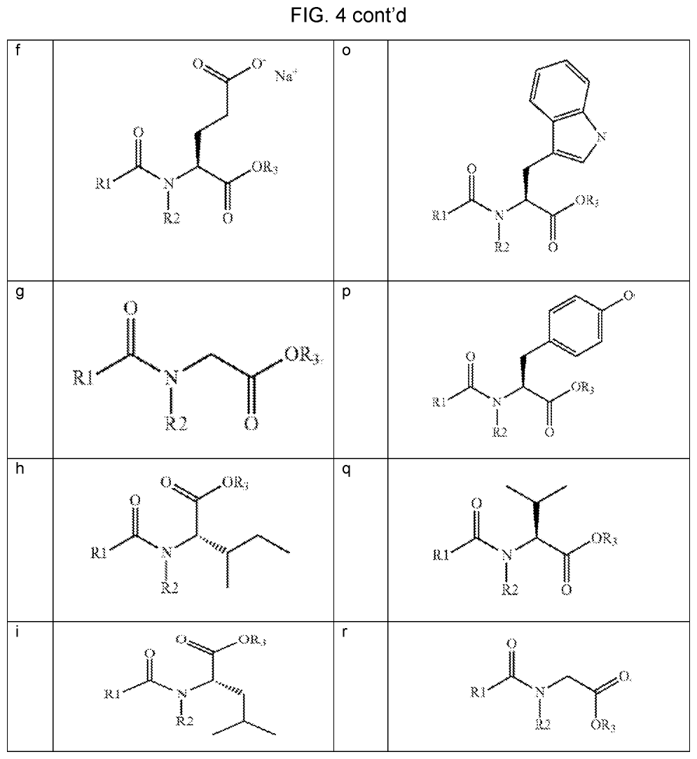

In particular embodiments the acylated FA-aa is selected from the group including formula (a), (b), (c), (d), (e), (f), (g), (h), (i), (j), (k), (l), (m), (n), (o), (p), (q), and (r), wherein R1 is an alkyl group including 5 to 19 carbon atoms, R2 is H (i.e. hydrogen) or CH₃ (i.e. methyl group), and R3 is H; or a salt or the free acid form thereof. Formulas (a), (b), (c), (d), (e), (f), (g), (h), (i), (j), (k), (l), (m), (n), (o), (p), (q), and (r) are provided in FIG. 4.

In particular embodiments, the acylated FA-aa can be selected from one or more of sodium N-dodecanoyl alaninate, N-dodecanoyl-L-alanine, sodium N-dodecanoyl isoleucinate, N-dodecanoyl-L-isoleucine, sodium N-dodecanoyl leucinate, N-dodecanoyl-L-leucine, sodium N-dodecanoyl methioninate, N-dodecanoyl-L-methionine, sodium N-dodecanoyl phenylalaninate, N-dodecanoyl-L-phenylalanine, sodium N-dodecanoyl prolinate, N-dodecanoyl-L-proline, sodium N-dodecanoyl tryptophanate, N-dodecanoyl-L-tryptophane, sodium N-dodecanoyl valinate, N-dodecanoyl-L-valine, sodium N-dodecanoyl sarcosinate, N-dodecanoyl-L-sarcosine, sodium N-oleoyl sarcosinate, sodium N-decyl leucine, sodium N-decanoyl alaninate, N-decanoyl-L-alanine, sodium N-decanoyl leucinate, N-decanoyl-L-leucine, sodium N-decanoyl phenylalaninate, N-decanoyl-L-phenylalanine, sodium N-decanoyl valinate, N-decanoyl-L-valine, sodium N-decanoyl isoleucinate, N-decanoyl-L-isoleucine, sodium N-decanoyl methioninate, N-decanoyl-L-methionine, sodium N-decanoyl prolinate, N-decanoyl-L-proline, sodium N-decanoyl threoninate, N-decanoyl-L-threonine, sodium N-decanoyl tryptophanate, N-decanoyl-L-tryptophane, sodium N-decanoyl sarcosinate, N-decanoyl-L-Sarcosine, N-dodecanoyl asparaginate, N-dodecanoyl-L-asparagine, sodium N-dodecanoyl aspartic acid, N-dodecanoyl-L-aspartic acid, sodium N-dodecanoyl cysteinate, N-dodecanoyl-L-cysteine, sodium N-dodecanoyl glutaminate, N-dodecanoyl-L-glutamine, sodium N-dodecanoyl glycinate, N-dodecanoyl-L-glycine, sodium N-dodecanoyl serinate, N-dodecanoyl-L-serine, sodium N-dodecanoyl threoninate, N-dodecanoyl-L-threonine, sodium N-dodecanoyl tyrosinate, N-dodecanoyl-L-tyrosine, sodium N-decanoyl asparaginate, N-decanoyl-L-asparagine, sodium N-decanoyl aspartic acid, N-decanoyl-L-aspartic acid, sodium N-decanoyl cysteinate, N-decanoyl-L-cysteine, sodium N-decanoyl glutaminate, N-decanoyl-L-glutamine, sodium N-decanoyl glycinate, N-decanoyl-L-glycine, sodium N-decanoyl serinate, N-decanoyl-L-serine, sodium N-decanoyl tyrosinate, N-decanoyl-L-tyrosine, sodium N-dodecanoyl asparaginate, sodium N-dodecanoyl glutamic acid, N-dodecanoyl-L-glutamic acid, sodium N-decanoyl glutamic acid, N-decanoyl-L-glutamic acid, Amisoft HS-11P (sodium Stearoyl Glutamate, Amisoft MS-11 (sodium Myristoyl Glutamate), Amisoft LS-11 (sodium Dodecanoyl Glutamate), Amisoft CS-11 (sodium Cocoyl Glutamate), sodium N-cocoyl glutamate, Amisoft HS-11P, Amisoft HS-11 P (sodium N-stearoyl glutamate), (sodium N-myristoyl glutamate)), (sodium N-dodecanoyl glutamate), and Amisoft HS-11 P.

The following acylated FA-aas are commercially available:

| | | Provider |
|---|---|---|
| | | (per 14- |
| Brand Name | Chemical Name | APR-2011) |
| Hamposyl L-95 | sodium N-dodecanoyl | Chattem |
| | sarcosinate | Chemicals |
| Hamposyl O | sodium N-oleoyl | Chattem |
| | sarcosinate | Chemicals |
| Hamposyl C | sodium N-cocoyl | Chattem |
| | sarcosinate | Chemicals |
| Hamposyl L-30 | sodium N-dodecanoyl | Chattem |
| | sarcosinate | Chemicals |
| Amisoft HS-11 P | sodium N-stearoyl | Ajinomoto |
| | glutamate | |
| Amisoft LS-11 | sodium N-dodecanoyl | Ajinomoto |
| | glutamate | |
| Amisoft CS-11 | sodium N-cocoyl | Ajinomoto |
| | glutamate | |
| Amisoft MS-11 | sodium N-myristoyl | Ajinomoto |
| | glutamate | |
| Amilite GCS-11 | sodium N-cocoyl | Ajinomoto |
| | glycinate | |

In particular embodiments the terms “fatty acid N-acylated amino acid”, “fatty acid acylated amino acid”, or “acylated amino acid” are used interchangeably herein and refer to an amino acid that is acylated with a fatty acid at its alpha-amino group.

Particular embodiments utilize vegetable matter with low solubility, or very low solubility. Particular embodiments utilize vegetable matter that is essentially water insoluble. In particular embodiments, solubility in water is defined as low to zero by the United States pharmacopeia (USP 32) according to the amount of water necessary for the dissolution of one part of solute: Low solubility: 100 to 1000 parts of water necessary for dissolution of one part of solute; very low solubility: 1000 to 10000 parts of water necessary; essentially water insoluble more than 10000 parts of water necessary. At a basic pH, however, SNAC and other modified amino acids and FA-aas described herein are water soluble. Thus, the administration benefits, as described herein could not be reasonably predicted. In particular embodiments, low water solubility can refer to a solubility in water or an aqueous solution of less than 1 mg/ml, less than 0.1 mg/ml, or less than 0.01 mg/ml.

In particular embodiments, N-acylated fatty amino acids act as absorption enhancing agents, thereby creating an administration benefit. Absorption enhancing agents refer to compounds that promote gastrointestinal absorption. Absorption enhancing agents can improve drug absorption by improving the solubility of the drug in the gastrointestinal tract or by enhancing membrane penetration, as compared to a formulation that does not include the absorption enhancing agents. Additional examples of absorption enhancing agents include surfactants, detergents, azones, pyrrolidones, glycols or bile salts.

In particular embodiments, N-acylated fatty amino acids act as bioavailability enhancing agents. Bioavailability refers to the fraction of active ingredient that is actually absorbed by a subject and reaches the bloodstream. In particular embodiments, bioavailability enhancing agents increase the fraction of active ingredient in the bloodstream or result in detection of active ingredient in the bloodstream earlier in time, as compared to a formulation that does not include the bioavailability enhancing agent.

In particular embodiments, additional administration benefits created by absorption enhancing agents and/or bioavailability enhancing agents include faster onset of action, higher peak concentrations, faster time to peak concentrations, increased subjective therapeutic efficacy, and/or increased objective therapeutic efficacy as compared to a control herbal composition or oral formulation based on the same, similar in all aspects but for inclusion of the absorption enhancing agents and/or bioavailability enhancing agents.

Embodiments utilizing absorption enhancing agents and/or bioavailability enhancing agents (e.g., and in particular embodiments, N-acylated fatty amino acids) can be beneficial because many orally administered herbal compositions designed to address various physiological conditions are inadequate because they are characterized by low bioavailability. Delayed onset of action presents challenges in clinical indications that require rapid therapeutic effect (e.g. pain and migraine); and low bioavailability requires patients to ingest significantly higher doses than would be required by alternative dosing forms. Particular embodiments disclosed herein provide oral formulations of herbal compositions with improved bioavailability and shorter time to onset of therapeutic effect.

In particular embodiments, the herbal compositions include botanical medicines. A botanical medicine refers to a plant, a plant component, and/or a plant extract that is used to treat illness or promote health.

In particular embodiments, the herbal compositions can be plant-based nutritional supplements. A nutritional supplement refers to a product that contains a dietary ingredient that is intended to add further nutritional value to the diet. Examples of plant-based nutritional supplements can include herbs and botanical products that add nutritional value to the diet.

As stated, in particular embodiments, N-acylated fatty amino acids act as subjective therapy enhancing agents. Subjective therapy enhancement refers to a noticeable alleviation of a symptom, as perceived by a subject. In particular embodiments, subjective therapy enhancing agents increase the alleviation of a symptom or alleviate a symptom more quickly, as compared to a formulation that does not include the subjective therapy enhancing agent.

In particular embodiments, N-acylated fatty amino acids act as objective therapy enhancing agents. Objective therapy enhancement refers to alleviation of a clinical measure, such as a nutritional deficiency detected by a blood or saliva assay or a test of wellness, as administered by a physician. In particular embodiments, objective therapy enhancing agents increase the alleviation of an objective clinical measure or result in alleviation more quickly, as compared to a formulation that does not include the objective therapy enhancing agent.

Particular embodiments include vegetable matter (e.g., an active component of Curcuma longa, Hypericum perforatum, and/or Polygonum cuspidatum) and an absorption enhancing agent and/or bioavailability enhancing agent. These embodiments can allow rapid absorption and higher bioavailability compared to vegetable matter ingested by currently available oral dosage forms.

In particular embodiments, carriers disclosed herein create administration benefits selected from: increased absorption, increased bioavailability, faster onset of action, higher peak concentrations, faster time to peak concentrations, increased subjective therapeutic efficacy, increased objective therapeutic efficacy, improved taste, and improved mouthfeel. Administration benefits related to increased absorption, increased bioavailability, faster onset of action, higher peak concentrations, faster time to peak concentrations, can alleviate adverse conditions more rapidly (for example, alleviation of pain). “Mouthfeel” refers to non-taste-related aspects of the pleasantness experienced by a person while ingesting (e.g., chewing or swallowing) an oral dosage form. Aspects of mouthfeel include the hardness and brittleness of a composition, whether the composition is chewy, gritty, oily, creamy, watery, sticky, easily dissolved, astringent, effervescent, and the like, and the size, shape, and form of the composition (tablet, powder, gel, etc.).

Herbal compositions can be manufactured for administration to a subject by adding vegetable matter, a carrier that provides an administration benefit, and one or more excipients, mixing, suspending, dissolving, blending, granulating, tableting, encapsulating, or performing other dosage-form-specific procedures, followed by packaging. For clarity, carriers contribute to providing an administration benefit. Excipients can, but need not, contribute to an administration benefit.

Particular embodiments include herbal compositions prepared as oral formulations. Exemplary oral formulations include capsules, coated tablets, edibles, elixirs, emulsions, gels, gelcaps, granules, gums, juices, liquids, oils, pastes, pellets, pills, powders, rapidly-dissolving tablets, sachets, semi-solids, sprays, solutions, suspensions, syrups, tablets, tinctures, etc.

Exemplary excipient classes include binders, buffers, chelators, coating agents, colorants, complexation agents, diluents (i.e., fillers), disintegrants, emulsifiers, flavoring agents, glidants, lubricants, preservatives, releasing agents, surfactants, stabilizing agents, solubilizing agents, sweeteners, thickening agents, wetting agents, and vehicles.

Binders are substances used to cause adhesion of powder particles in granulations. Exemplary binders include acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, and polyethylene glycol.

Colorants may be included in the oral formulations to impart color to the formulation. Exemplary colorants include grape skin extract, beet red powder, beta carotene, annato, carmine, turmeric, and paprika. Additional colorants include FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide.

Diluents can enhance the granulation of oral formulations. Exemplary diluents include microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycine.

Disintegrants also may be included in the oral formulations in order to facilitate dissolution. Disentegrants, including permeabilising and wicking agents, are capable of drawing water or saliva up into the oral formulations which promotes dissolution from the inside as well as the outside of the oral formulations. Such disintegrants, permeabilising and/or wicking agents that may be used include starches, such as corn starch, potato starch, pre-gelatinized and modified starches thereof, cellulosic agents, such as Ac-di-sol, montmorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose. Dissolution of the oral formulations may be facilitated by including relatively small particles sizes of the ingredients used.

Exemplary dispersing or suspending agents include acacia, alginate, dextran, fragacanth, gelatin, hydrogenated edible fats, methylcellulose, polyvinylpyrrolidone, sodium carboxymethyl cellulose, sorbitol syrup, and synthetic natural gums.

Exemplary emulsifiers include acacia and lecithin.

Flavorants are natural or artificial compounds used to impart a pleasant flavor and often odor to oral formulations. Exemplary flavorants include, natural and synthetic flavor oils, flavoring aromatics, extracts from plants, leaves, flowers, and fruits and combinations thereof. Such flavorants include anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wildberry, date, blueberry, kiwi, strawberry, raspberry, cherry, plum, pineapple, and apricot. In particular embodiments, flavorants that may be used include natural berry extracts and natural mixed berry flavor, as well as citric and malic acid.

Glidants improve the flow of powder blends during manufacturing and minimize oral formulation weight variation. Exemplary glidants include silicon dioxide, colloidal or fumed silica, magnesium stearate, calcium stearate, stearic acid, cornstarch, and talc.

Lubricants are substances used in oral formulations that reduce friction during composition compression. Exemplary lubricants include stearic acid, calcium stearate, magnesium stearate, zinc stearate, talc, mineral and vegetable oils, benzoic acid, poly(ethylene glycol), glyceryl behenate, stearyl fumarate, and sodium lauryl sulfate.

Exemplary preservatives include methyl p-hydroxybenzoates, propyl p-hydroxybenzoates, and sorbic acid.

Exemplary sweeteners include aspartame, dextrose, fructose, high fructose corn syrup, maltodextrin, monoammonium glycyrrhizinate, neohesperidin dihydrochalcone, potassium acesulfame, saccharin sodium, stevia, sucralose, and sucrose.

Particular embodiments include swallowable compositions. Swallowable compositions are those that do not readily dissolve when placed in the mouth and may be swallowed whole without chewing or discomfort. U.S. Pat. Nos. 5,215,754 and 4,374,082 describe methods for preparing swallowable compositions. In particular embodiments, swallowable compositions may have a shape containing no sharp edges and a smooth, uniform and substantially bubble free outer coating.

To prepare swallowable compositions, each of the ingredients may be combined in intimate admixture with a suitable carrier according to conventional compounding techniques. In particular embodiments of the swallowable compositions, the surface of the compositions may be coated with a polymeric film. Such a film coating has several beneficial effects. First, it reduces the adhesion of the compositions to the inner surface of the mouth, thereby increasing the subject's ability to swallow the compositions. Second, the film may aid in masking the unpleasant taste of certain ingredients. Third, the film coating may protect the compositions from atmospheric degradation. Polymeric films that may be used in preparing the swallowable compositions include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia.

In particular embodiments, the oral formulations may include chewable compositions. Chewable compositions are those that have a palatable taste and mouthfeel, are relatively soft and quickly break into smaller pieces and begin to dissolve after chewing such that they are swallowed substantially as a solution.

U.S. Pat. No. 6,495,177 describes methods to prepare chewable compositions with improved mouthfeel. U.S. Pat. No. 5,965,162, describes kits and methods for preparing comestible units which disintegrate quickly in the mouth, especially when chewed.

In order to create chewable compositions, certain ingredients should be included to achieve the attributes just described. For example, chewable compositions should include ingredients that create pleasant flavor and mouthfeel and promote relative softness and dissolvability in the mouth. The following discussion describes ingredients that may help to achieve these characteristics.

Sugars such as white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Fondant or gums such as gelatin, agar, arabic gum, guar gum, and carrageenan may be added to improve the chewiness of the compositions. Fatty materials that may be used include vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used to enhance the texture, the mouthfeel, or both of chewable compositions. By “enhance the texture” it is meant that the alkyl polysiloxane improves one or more of the stiffness, the brittleness, and the chewiness of the chewable composition, relative to the same preparation lacking the alkyl polysiloxane. By “enhance the mouthfeel” it is meant that the alkyl polysiloxane reduces the gritty texture of the chewable composition once it has liquefied in the mouth, relative to the same preparation lacking the alkyl polysiloxane.

Alkyl polysiloxanes generally include a silicon and oxygen-containing polymeric backbone with one or more alkyl groups pending from the silicon atoms of the back bone. Depending upon their grade, they can further include silica gel. Alkyl polysiloxanes are generally viscous oils. Exemplary alkyl polysiloxanes that can be used in swallowable, chewable or dissolvable compositions include monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which includes 30% simethicone USP. Simethicone USP includes not less than 90.5% by weight (CH₃)₃—Si{OSi(CH₃)₂}CH₃ in admixture with 4.0% to 7.0% by weight SiO₂.

To prevent the stickiness that can appear in some chewable compositions and to facilitate conversion of the active ingredients to emulsion or suspension upon taking, the compositions may further include emulsifiers such as glycerin fatty acid ester, sorbitan monostearate, sucrose fatty acid ester, lecithin and mixtures thereof. In particular embodiments, one or more of such emulsifiers may be present in an amount of 0.01% to 5.0%, by weight of the administered formulations. If the level of emulsifier is lower or higher, in particular embodiments, an emulsification cannot be realized, or wax value will rise.

Liquid preparations for oral administration may take the form of, for example, solutions, syrups or suspensions, or they may be presented as a dry product for reconstitution with water or other suitable vehicles before use.

In addition to those described above, any appropriate fillers and excipients may be utilized in preparing the swallowable, chewable and/or dissolvable compositions or any other oral formulation described herein so long as they are consistent with the described objectives.

Oral formulations also include edibles. Edibles refer to any product that can be consumed as a food or a drink. In some cases, edibles are made by infusion of plant extracts into a foodstuff. Examples of edible foods appropriate for use include candy, a candy bar, bread, a brownie, cake, cheese, chocolate, cocoa, a cookie, gummy candy, a lollipop, a mint, a pastry, peanut butter, popcorn, a protein bar, rice cakes, yogurt, etc. While technically not edible, gums can also be used. Examples of edible drinks include beer, juice, flavored milk, flavored water, liquor, milk, punch, a shake, soda, tea, and water. In particular embodiments, edibles are made by combining a plant extract with ingredients used to make an edible. Examples include butters and oils. Exemplary oils include coconut oil, grape seed oil, olive oil, palm oil, papaya seed oil, peanut oil, sesame oil, sprouted wheat oil, wheat germ oil, or any combination thereof.

Oral formulations can be individually wrapped or packaged as multiple units in one or more packages, cans, vials, blister packs, or bottles of any size. Doses are sized to provide therapeutically effective amounts.

In particular embodiments, the oral formulations include vegetable matter (e.g., an active component of Curcuma longa, Hypericum perforatum, Polygonum cuspidatum, and/or Vitis spp.) of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

In particular embodiments, the oral formulations include carrier of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

In particular embodiments, the oral formulations include excipient of at least 0.1% w/v or w/w of the oral formulation; at least 1% w/v or w/w of oral formulation; at least 10% w/v or w/w of oral formulation; at least 20% w/v or w/w of oral formulation; at least 30% w/v or w/w of oral formulation; at least 40% w/v or w/w of oral formulation; at least 50% w/v or w/w of oral formulation; at least 60% w/v or w/w of oral formulation; at least 70% w/v or w/w of oral formulation; at least 80% w/v or w/w of oral formulation; at least 90% w/v or w/w of oral formulation; at least 95% w/v or w/w of oral formulation; or at least 99% w/v or w/w of oral formulation.

In particular embodiments, 10 g of dried plant extract may be used in 150 ml of water. This may give an effective concentration of between 1 and 99% (w/w) plant extract, between 2 and 80% (w/w) plant extract, and between 5 and 50% (w/w) plant extract.

Excipients are commercially available from companies such as Aldrich Chemical Co., FMC Corp, Bayer, BASF, Alexi Fres, Witco, Mallinckrodt, Rhodia, ISP, and others.

Additional information can be found in WADE & WALLER, HANDBOOK OF PHARMACEUTICAL EXCIPIENTS (2nd ed. 1994) and Remington's Pharmaceutical Sciences, 18th Ed. Mack Printing Company, 1990. Moreover, formulations can be prepared to meet sterility, pyrogenicity, general safety, and purity standards as required by U.S. FDA and/or other relevant foreign regulatory agencies.

Herbal compositions disclosed herein can be used to treat subjects (humans, veterinary animals (dogs, cats, reptiles, birds, etc.), livestock (horses, cattle, goats, pigs, chickens, etc.), and research animals (monkeys, rats, mice, fish, etc.)). Treating subjects includes providing therapeutically effective amounts. Therapeutically effective amounts include those that provide effective amounts, prophylactic treatments, and/or therapeutic treatments.

An “effective amount” is the amount of an herbal composition necessary to result in a desired physiological change in a subject. Effective amounts are often administered for research purposes. Representative effective amounts disclosed herein can reduce pain perception in an animal model (chronic pain, acute pain, visceral pain), reduce insomnia in an animal model, reduce symptoms of inflammation in an animal model, improve wound healing in an animal model, improve digestion in an animal model, reduce anxiety in an animal model, and/or reduce symptoms of asthma in an animal model.

A “prophylactic treatment” includes a treatment administered to a subject who does not display signs or symptoms of a disease or nutritional deficiency, or displays only early signs or symptoms of a disease or nutritional deficiency, such that treatment is administered for the purpose of diminishing, preventing, or decreasing the risk of developing the disease or nutritional deficiency further. Thus, a prophylactic treatment functions as a preventative treatment against the development of diseases or nutritional deficiencies.

As one example of a prophylactic treatment, an oral formulation disclosed herein can be administered to a subject who is at risk of developing insomnia. An effective prophylactic treatment of insomnia occurs when the number of incidences of insomnia per month experienced by a subject is reduced by at least 10% or in particular embodiments, by 25%.

As another example of a prophylactic treatment, an oral formulation disclosed herein can be administered to a subject who is at risk of suffering from pain. An effective prophylactic treatment of pain occurs when the occurrence of the pain is reduced by at least 10%, or in particular embodiments, by 25% as measured by a standard subjective or objective pain assessment.

As another example of a prophylactic treatment, an oral formulation disclosed herein can be administered to a subject who is at risk of depression. An effective prophylactic treatment of depression occurs when the severity of depression is reduced by at least 10%, or in particular embodiments, by 25% as measured by a standard subjective or objective depression assessment.

As another example of a prophylactic treatment, an oral formulation disclosed herein can be administered to a subject who is at risk of inflammation. An effective prophylactic treatment of inflammation occurs when the severity of inflammation is reduced by at least 10%, or in particular embodiments, by 25% as measured by a standard subjective or objective inflammation assessment.

A “therapeutic treatment” includes a treatment administered to a subject who has a disease or nutritional deficiency and is administered to the subject for the purpose of curing or reducing the severity of the disease or nutritional deficiency.

As one example of a therapeutic treatment, an oral formulation disclosed herein can be administered to a subject who has inflammatory bowel disease. An effective therapeutic treatment of inflammatory bowel disease occurs when the severity of symptoms of inflammatory bowel disease are reduced or relieved completely.

Another example of a therapeutic treatment includes administration of an oral formulation disclosed herein to a subject who has anxiety. An effective therapeutic treatment of anxiety occurs when the severity of the anxiety is reduced or relieved completely and/or more quickly, as measured by a standard subjective or objective anxiety assessment.

Another example of a therapeutic treatment includes administration of an oral formulation disclosed herein to a subject who has inflammation. An effective therapeutic treatment of inflammation occurs when the severity of the inflammation is reduced or relieved completely and/or more quickly, as measured by a standard subjective or objective inflammation assessment.

Another example of a therapeutic treatment includes administration of an oral formulation disclosed herein to a subject who has depression. An effective therapeutic treatment of depression occurs when the severity of the depression is reduced or relieved completely and/or more quickly, as measured by a standard subjective or objective depression assessment.

Another example of a therapeutic treatment includes administration of an oral formulation disclosed herein to a subject who has cancer. An effective therapeutic treatment of cancer occurs when the severity of the cancer is reduced or relieved completely and/or more quickly, as measured by a standard subjective or objective cancer assessment.

Therapeutic treatments can be distinguished from effective amounts based on the presence or absence of a research component to the administration. As will be understood by one of ordinary skill in the art, however, in human clinical trials effective amounts, prophylactic treatments and therapeutic treatments can overlap.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by the subject, a physician, veterinarian, or researcher taking into account parameters such as physical, physiological and psychological factors including target, body weight, condition, previous or concurrent therapeutic interventions, and/or idiopathy of the subject.

Useful doses can range from 0.1 to 5 μg/kg or from 0.5 to 1 μg/kg. In particular embodiments, a dose of vegetable matter, an active component of a plant, and/or a plant extract can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg or from 0.5 to 1 mg/kg. In particular embodiments, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg or more.

In particular embodiments, useful doses include weight of vegetable matter (e.g., an active component of a plant or a plant extract) per body weight of a subject. In particular embodiments, useful doses can range from 0.1 mg/kg to 100 mg/kg or from 0.5 mg/kg to 50 mg/kg. In particular embodiments, useful doses include 0.5 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, or more of vegetable matter per body weight of a subject.

In particular embodiments, useful doses include weight of carrier (e.g., SNAC) per body weight of a subject. In particular embodiments, useful doses can range from 0.1 mg/kg to 100 mg/kg or from 0.5 mg/kg to 50 mg/kg. In particular embodiments, useful doses include 0.5 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, or more of carrier per body weight of a subject.

In particular embodiments, total dose volume can range from 0.25 mL to 30 mL or from 0.5 mL to 20 mL. In particular embodiments, a total dose volume can include 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, or more.

Dose concentration can be expressed as weight of vegetable matter (e.g., an active component of a plant or a plant extract) or active ingredient per dose volume (e.g., mg active pharmaceutical ingredient (API)/mL). In particular embodiments, dose concentration can range from 1 mg/mL to 100 mg/mL or from 5 mg/mL to 50 mg/mL. In particular embodiments, a dose concentration can include 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, or more.

Dose concentration can be expressed as weight of carrier (e.g., SNAC) per dose volume (e.g., mg SNAC/mL). In particular embodiments, dose concentration can range from 1 mg/mL to 500 mg/mL or from 50 mg/mL to 300 mg/mL. In particular embodiments, a dose concentration can include 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, or more.

In particular embodiments, the ratio of carrier to vegetable matter (e.g., an active component of a plant or a plant extract) or active ingredient (w/w) can range from 1:1 to 100:1 or from 1:1 to 20:1. In particular embodiments, the ratio can include 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, 100:1, or more. In particular embodiments, the ratio can be 10:1.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

One or more active component(s) and/or plant extract(s) can be administered simultaneously or within a selected time window, such as within 10 minutes, 1 hour, 3 hour, 10 hour, 15 hour, 24 hour, or 48 hour time windows or when the complementary active agent(s) is within a clinically-relevant therapeutic window.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXEMPLARY EMBODIMENTS

1. An herbal composition with improved bioavailability formulated for oral delivery including (i) capsaicin, reserpine, vinblastine, hesperidin, naringin, rutin, quercitrin, curcumin, hypericin, resveratrol, catechin eugenol, limonene, or linalool and (ii) N-[8-(2-hydroxybenzoyl) amino] caprylate.
2. An herbal composition with improved bioavailability formulated for oral delivery including (i) vegetable matter derived from Curcuma longa, Hypericum perforatum, Polygonum cuspidatum, Vitis spp., Camellia sinsensis, Theobroma cacao, Capsicum spp., Rauwolfia vomitoria, Rauwolfia serpentina, Vinca spp., Citrus spp., Rheum rhabarbarum. Fagopyrum tataricum, Syzygium aromaticum, Lavandula spp., or Mentha spp., and (ii) an N-acylated fatty amino acid or salt thereof.
3. An herbal composition with improved bioavailability formulated for oral delivery including (i) an active component of Curcuma longa, Hypericum perforatum, Polygonum cuspidatum, Vitis spp., Camellia sinsensis, Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., or Mentha spp., wherein the active component has low water solubility and (ii) an N-acylated fatty amino acid or salt thereof.
4. An herbal composition with improved bioavailability formulated for oral delivery including (i) an extract of Curcuma longa, Hypericum perforatum, Polygonum cuspidatum, Vitis spp., Camellia sinsensis, Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., or Mentha spp., and (ii) an N-acylated fatty amino acid or salt thereof.
5. The herbal composition of any of embodiments 1-4 further comprising vegetable matter derived from Calophyllum brasiliense, Calophyllum caledonicurn, Calophyllum inophyllum, Calophyllum soulattri, Uncaria tomentosa, Thymus vulgaris, Matricaria recutita, Salix alba, Calendula officinalis, Usnea barbata, Ligusticum porterii-osha, Gaultheria procumbens, Vaccinium myrtillus, Melissa officinalis, Allium sativum, Krameria triandra, Punica granatum, Viburnum plicatum, Duboisia hopwoodii, Asclepias syriaca, Cannabis sativa, Cannabis indica, Cannabis ruderalis and/or Acer spp, or an extract thereof.
6. The herbal composition of any of embodiments 2-5 including capsaicin, reserpine, vinblastine, hesperidin, naringin, rutin, quercitrin, curcumin, hypericin, resveratrol, catechin eugenol, limonene, or linalool.
7. An herbal composition comprising (i) a polyphenol, alkaloid, glycoside, or terpene that is derived from a plant and has low water solubility and (ii) an N-acylated fatty amino acid.
8. The herbal composition of any of embodiments 2-7 wherein the N-acylated fatty amino acid includes one or more of Compounds I-XXXV (FIG. 3), or Compounds a-r (FIG. 4).
9. The herbal composition of any of embodiments 2-8 wherein the N-acylated fatty amino acid includes monosodium-N-salicyloyl-8-aminocaprylate, disodium-N-salicyloyl-8-aminocaprylate, or N-(salicyloyl)-8-aminocaprylic acid.
10. The herbal composition of any of embodiments 2-9 wherein the N-acylated fatty amino acid or a salt thereof includes

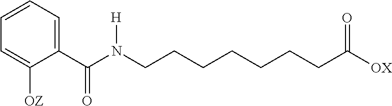

wherein X and Z are independently H, a monovalent cation, a divalent metal cation, or an organic cation.
11. The herbal composition of embodiment 10, wherein the monovalent cation is sodium or potassium.
12. The herbal composition of embodiment 10 or 11, wherein the divalent metal cation is calcium or magnesium.
13. The herbal composition of any of embodiments 10-12, wherein the organic cation is ammonium or tetramethylammonium.
14. The herbal composition of any of embodiments 10-13, wherein X is H.
15. The herbal composition of any of embodiments 10-13, wherein X is a monovalent cation including sodium or potassium.
16. The herbal composition of any of embodiments 10-13, wherein X is a divalent metal cation including calcium or magnesium.
17. The herbal composition of any of embodiments 10-13, wherein X is an organic cation including ammonium or tetramethylammonium.
18. The herbal composition of any of embodiments 10-17, wherein Z is H.
19. The herbal composition of any of embodiments 10-17, wherein Z is a monovalent cation including sodium or potassium.
20. The herbal composition of any of embodiments 10-17, wherein Z is a divalent cation including calcium or magnesium.
21. The herbal composition of embodiment 10, wherein X is H and Z is H.
22. The herbal composition of embodiment 10, wherein X is H and Z is sodium.
23. The herbal composition of embodiment 10, wherein X is sodium and Z is sodium.
24. The herbal composition of any of embodiments 1-23 wherein the N-acylated fatty amino acid or salt thereof provides an administration benefit.
25. The herbal composition of embodiment 24 wherein the administration benefit is a dose-dependent administration benefit.
26. The herbal composition of embodiment 25 wherein the dose-dependent administration benefit is at a dose of 100-200 mg.
27. The herbal composition of any of embodiments 24-26 wherein the administration benefit includes one or more of increased absorption of a measured component of vegetable matter, increased bioavailability of a measured component of vegetable matter, faster onset of action of a measured component of vegetable matter, higher peak concentrations of a measured component of vegetable matter, faster time to peak concentrations of a measured component of vegetable matter, increased subjective therapeutic efficacy, increased objective therapeutic efficacy, improved taste, and improved mouthfeel as compared to a control composition without the N-acylated fatty amino acid.
28. The herbal composition of any of embodiments 1-27 including a botanical medicine.
29. The herbal composition of any of embodiments 1-28 including a nutritional supplement.
30. The herbal composition of any of embodiments 1-29 including a botanical product.
31. The herbal composition of any of embodiments 1-30 including a surfactant, detergent, azone, pyrrolidone, glycol or bile salt.
32. The herbal composition of any of embodiments 1-31 including a therapeutically effective amount of the vegetable matter.
33. The herbal composition of embodiment 32 wherein the therapeutically effective amount treats a symptom of acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, and withdrawal.
34. The herbal composition of any of embodiments 1-33 including vitamins or minerals.
35. The herbal composition of any of embodiments 1-33 including vitamins and minerals.
36. The herbal composition of embodiment 34 or 35 wherein the vitamins are selected from one or more of Vitamin A, Vitamin B1, Vitamin B6, Vitamin B12, Vitamin C, Vitamin D, Vitamin E, or Vitamin K.
37. The herbal composition of embodiment 34 or 35 wherein the minerals are selected from one or more of calcium, chromium, iodine, iron, magnesium, selenium and/or zinc.
38. An oral formulation including an herbal composition of any of embodiments 1-37.
39. An oral formulation of embodiment 38 wherein the oral formulation is swallowable or chewable.
40. An oral formulation of embodiment 38 or 39 wherein the oral formulation is liquid or solid.
41. An oral formulation of any of embodiments 38-40 wherein the oral formulation is a solution, suspension, or spray.
42. An oral formulation of any of embodiments 38-40 wherein the oral formulation is a tablet, capsule or sachet.
43. An oral formulation of any of embodiments 38-42 wherein the oral formulation is flavored.
44. A method of preparing an oral formulation of an herbal composition having improved bioavailability, wherein the method includes adding an absorption enhancer to the oral formulation of the herbal composition and wherein the oral formulation of the herbal composition has improved bioavailability compared to an oral formulation of the herbal composition without an absorption enhancer.
45. The method of embodiment 44, wherein the absorption enhancer is an N-acylated fatty amino acid or a salt thereof.
46. The method of embodiment 45, wherein the N-acylated fatty amino acid or a salt thereof includes

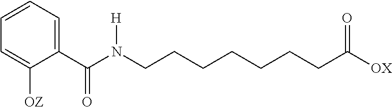

wherein X and Z are independently H, a monovalent cation, a divalent metal cation, or an organic cation.
47. The method of embodiment 45, wherein the N-acylated fatty amino acid is selected from monosodium-N-salicyloyl-8-aminocaprylate, disodium-N-salicyloyl-8-aminocaprylate, and N-(salicyloyl)-8-aminocaprylic acid.
48. A method of treating a subject in need thereof including administering a therapeutically effective amount of a composition of any of embodiments 1-37 to the subject thereby treating the subject in need thereof.
49. A method of embodiment 48 wherein the therapeutically effective amount provides an effective amount, a prophylactic treatment, and/or a therapeutic treatment.
50. A method of reducing or eliminating one or more symptoms of a disease or disorder in a human subject,
   wherein said method includes delivering a therapeutically effective amount of a composition of any of embodiments 1-37 to the subject, thereby reducing or eliminating one or more symptoms of the disease or disorder, and
   wherein said disease or disorder is acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.

### EXAMPLES

Oral dosage forms of herbal compositions providing improved bioeffect (e.g., bioavailability) and shortened time to onset of effect. The increased intensity of the observed effect and the shortened time to onset of action are indicative of improved bioavailability. Considering the wealth of medical conditions potentially benefiting from herbal remedies, a significant unmet need exists for a faster-acting product that provides improved bioavailability in an oral format. Traditional pharmaceutical dosage forms of many herbal compositions are challenged by low bioavailability, and prolonged time to onset of action. The present disclosure addresses the shortcomings of orally ingesting herbal compositions that have poor bioavailability.

### Example 1

Exemplary Formulations. Solution formulation. Vegetable matter (e.g., a plant extract or an active component of a plant such as curcumin) and one or more N-acylated fatty amino acids are combined in an aqueous/organic solvent mixture. The resulting blend is stirred vigorously for an hour. If solution is incomplete, a surfactant can be added and stirring can be continued to prepare the final formulation.

Suspension formulation. Vegetable matter (e.g., a plant extract or an active component of a plant such as curcumin) and one or more N-acylated fatty amino acids are combined in water, an aqueous/organic solvent mixture or an organic solvent mixture. The resulting blend can be stirred to effect suspension.

Solution formulation. Vegetable matter (e.g., a plant extract or an active component of a plant such as curcumin) and one or more absorption enhancing agents are combined in an aqueous/organic solvent mixture. The resulting blend is stirred vigorously for an hour. If solution is incomplete, a surfactant can be added and stirring can be continued to prepare the final formulation.

Suspension formulation. Vegetable matter (e.g., a plant extract or an active component of a plant such as curcumin) and one or more absorption enhancing agents are combined in water, an aqueous/organic solvent mixture or an organic solvent mixture. The resulting blend can be stirred to effect suspension.

Gelcap composition. A suspension formulation or solution formulation can be filled into a gelcap to contain up to 1 g of vegetable matter. The gelcap can be treated with an enteric coat or used without a coating.

Tablet/capsule composition. The solution formulation and the suspension formulation can be dried by evaporation, lyophilization, or spray drying. The resultant dry product can be combined with tableting excipients and compressed into tablets or caplets to contain up to 1 g of vegetable matter. Alternatively, the dry product can be filled into capsules.

### Example 2

Onset and duration of action of orally administered cannabis/SNAC composition. This study was designed to assess the utility of SNAC in enabling a rapid-acting oral form of an herbal composition.

Selection of Participants. Six study participants were recruited to ingest herbal compositions and record the onset, duration, and intensity of, in this example, cannabis-induced euphoria and/or dysphoria. Study participants took part in two separate tests: 1) use of a control substance, which included liquid cannabis extract dissolved in aqueous ethanol, and 2) use of a test substance, which included the liquid cannabis extract dissolved in aqueous ethanol, as well as SNAC.

Formulations. The selected cannabis concentrate is commercially available and was provided to participants in an ethanol solution. The concentrate contains 8 mg THC per dose. It was selected because it contains a high percentage of THC, which provides a noticeable effect on user-reported “euphoria”. Aqueous ethanol was used as solvent because it effectively dissolves cannabis extract, as well as SNAC.

Methods. For the Control experiment, each participant mixed the cannabis concentrate with 15 ml (one tablespoon) of aqueous ethanol, and immediately swallowed the mixture.

For the Test experiment, each participant mixed the cannabis concentrate with a pre-mixed solution of aqueous ethanol and 200 mg SNAC, and immediately swallowed the dissolved mixture.

For both the Control experiment and the Test experiment, each participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-10. Table 1 shows descriptions of euphoria and dysphoria levels for each scale value.

TABLE 1
Scale Values for Reporting Euphoria and Dysphoria

| Scale Value | Description |
|---|---|
| 0 | No observed effect |
| 1-2 | Mild observed effect; possibly psychological |
| 3-4 | Definite but mild effect |
| 5-6 | Definite substantial effect |
| 7-8 | Strong effect |
| 9-10 | Intense effect |

TABLE 2
Control Experiment (n = 6)

| | Time from | Observed | Observed |
|---|---|---|---|
| Actual Time | Start | “Euphoria” | “Dysphoria” |
| 12:00 PM | 0:00 | (0 - 10) | (0 - 10) |
| 12:15 PM | 0:15 | 0.17 | 0.00 |
| 12:30 PM | 0:30 | 0.50 | 0.00 |
| 12:45 PM | 0:45 | 0.83 | 0.17 |
| 1:00 PM | 1:00 | 1.33 | 0.17 |
| 1:15 PM | 1:15 | 1.67 | 0.50 |
| 1:30 PM | 1:30 | 1.83 | 0.67 |
| 1:45 PM | 1:45 | 1.83 | 0.83 |
| 2:00 PM | 2:00 | 2.00 | 0.50 |
| 2:15 PM | 2:15 | 2.17 | 0.50 |
| 2:30 PM | 2:30 | 1.83 | 0.33 |
| 2:45 PM | 2:45 | 1.67 | 0.33 |
| 3:00 PM | 3:00 | 2.17 | 0.33 |
| 3:15 PM | 3:15 | 1.33 | 0.17 |
| 3:30 PM | 3:30 | 1.17 | 0.00 |
| 3:45 PM | 3:45 | 1.00 | 0.00 |
| 4:00 PM | 4:00 | 1.00 | 0.00 |
| 4:15 PM | 4:15 | 0.83 | 0.00 |
| 4:30 PM | 4:30 | 0.67 | 0.00 |
| 4:45 PM | 4:45 | 0.50 | 0.00 |
| 5:00 PM | 5:00 | 0.17 | 0.00 |

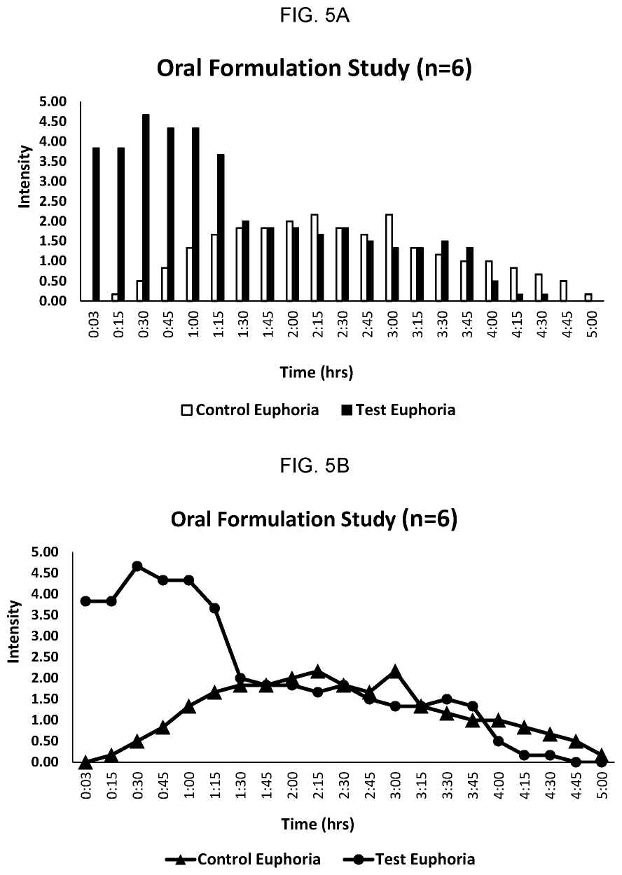

Results. The results shown below are the average scale values obtained for all six participants (also shown in FIGS. 5A and 5B).

TABLE 3
Test Experiment (n = 6)

| | Time from | Observed | Observed |
|---|---|---|---|
| Actual Time | Start | “Euphoria” | “Dysphoria” |
| 12:00 PM | 0:00 | (0 - 10) | (0 - 10) |
| 12:03 PM | 0:03 | 3.83 | 0.67 |
| 12:15 PM | 0:15 | 3.83 | 0.67 |
| 12:30 PM | 0:30 | 4.67 | 0.83 |
| 12:45 PM | 0:45 | 4.33 | 0.50 |
| 1:00 PM | 1:00 | 4.33 | 0.50 |
| 1:15 PM | 1:15 | 3.67 | 0.67 |
| 1:30 PM | 1:30 | 2.00 | 0.17 |
| 1:45 PM | 1:45 | 1.83 | 0.17 |
| 2:00 PM | 2:00 | 1.83 | 0.00 |
| 2:15 PM | 2:15 | 1.67 | 0.00 |
| 2:30 PM | 2:30 | 1.83 | 0.00 |
| 2:45 PM | 2:45 | 1.50 | 0.00 |
| 3:00 PM | 3:00 | 1.33 | 0.17 |
| 3:15 PM | 3:15 | 1.33 | 0.17 |
| 3:30 PM | 3:30 | 1.50 | 1.00 |
| 3:45 PM | 3:45 | 1.33 | 0.00 |
| 4:00 PM | 4:00 | 0.50 | 0.00 |
| 4:15 PM | 4:15 | 0.17 | 0.00 |
| 4:30 PM | 4:30 | 0.17 | 0.00 |
| 4:45 PM | 4:45 | 0.00 | 0.00 |
| 5:00 PM | 5:00 | 0.00 | 0.00 |

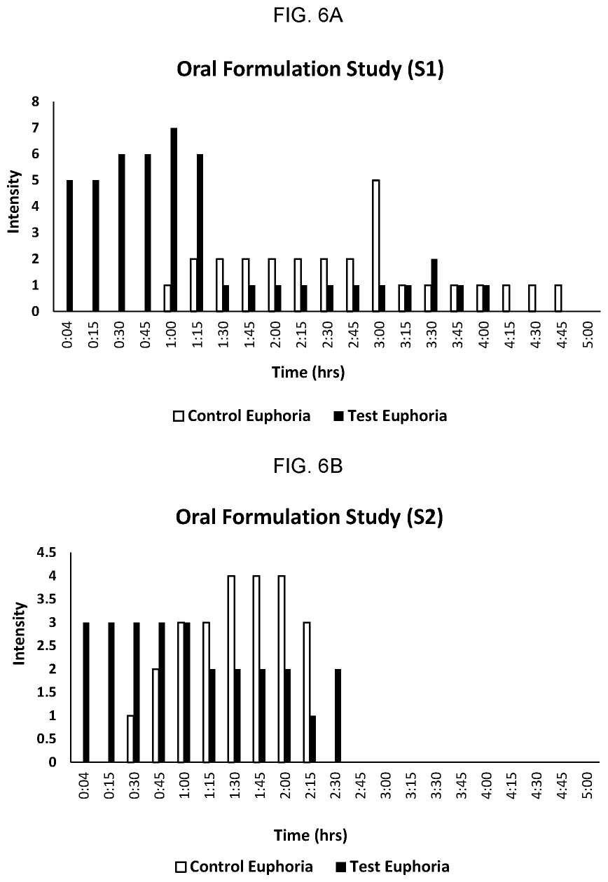

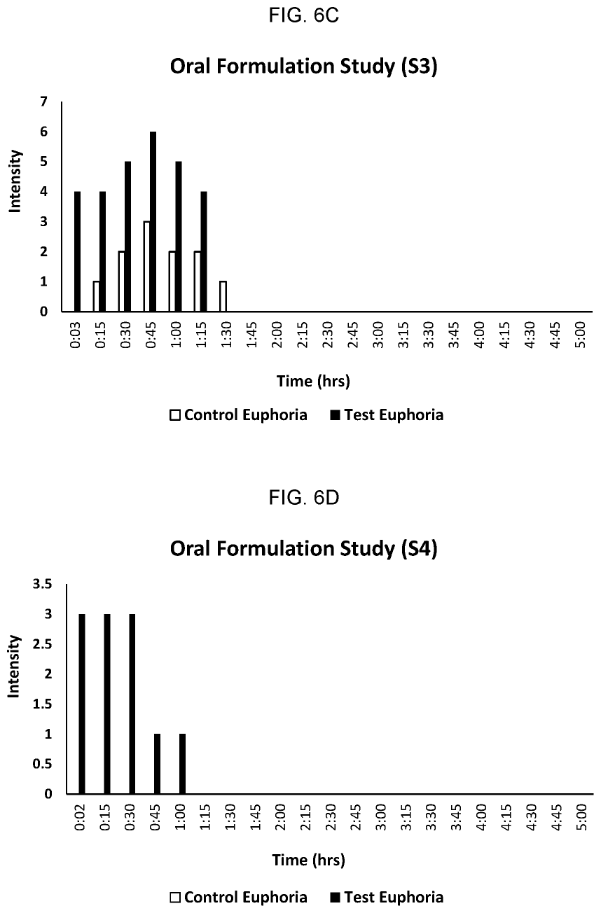

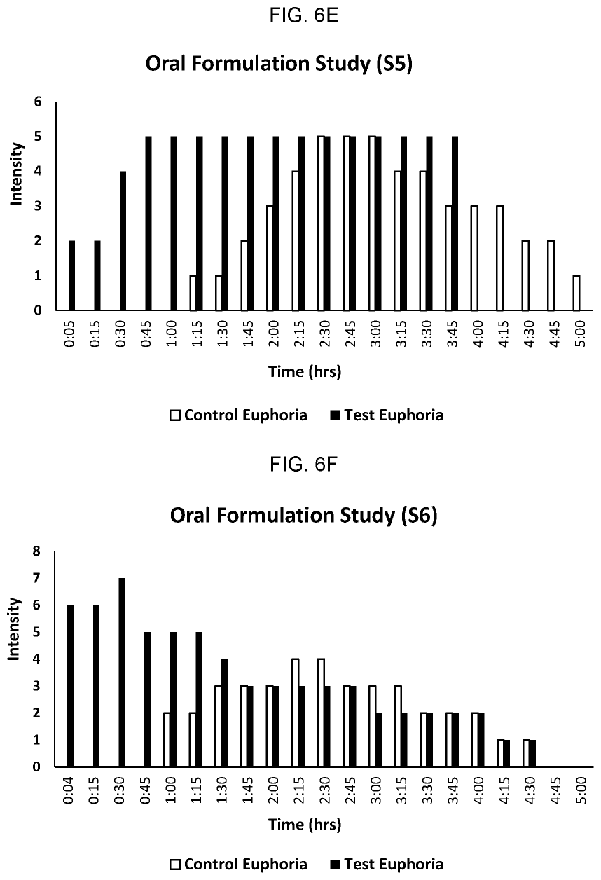

Onset: All six participants reported euphoria within five minutes of ingesting the cannabis/SNAC formulation (Test), with the time of onset ranging between two and five minutes. In contrast, the first time-point of euphoria reported by participants after ingestion of the cannabis-only formulation (Control) was fifteen minutes post-ingestion, with the time of onset ranging between fifteen minutes and one hour, fifteen minutes (see FIGS. 6A-6F for individual participant results). By fifteen minutes post-ingestion, the average reported euphoria scale value was 3.8 for the cannabis/SNAC formulation (Test). In contrast, fifteen minutes after ingestion of the cannabis-only formulation (Control), the average reported euphoria scale value was 0.17 (see FIGS. 5A-5B for averages at each time-point).

Intensity: The average peak euphoria scale value after ingestion of the cannabis/SNAC formulation (Test) was 4.7, which occurred thirty minutes post-ingestion. In contrast, the highest average euphoria scale value after ingestion of the cannabis-only formulation (Control) was 2.2, which was at the two hour, fifteen minute time-point (see FIGS. 5A and 5B). Therefore, ingestion of the cannabis/SNAC formulation led to a higher peak intensity of euphoria, which occurred an average of one hour and forty-five minutes faster than when the cannabis-only formulation was ingested. The intensity of observed dysphoria was minimal for both the Test and Control, with a peak average scale value of 0.83 for both experiments.

Duration: The results indicate that the addition of an absorption enhancer does not shorten the action of cannabis.

In summary, adding an absorption enhancer, such as SNAC, in an oral dosage formulation of an herbal composition provides faster onset of action and higher intensity of action at peak activity level. Moreover, the absorption enhancer has no effect on the duration of action of an herbal composition.

### Example 3

Onset and duration of action of orally administered herbal composition/SNAC composition at a low SNAC dose. This study was designed to assess the utility of SNAC in enabling a rapid-acting oral form of herbal compositions at a low dose.

Selection of Participants. Three study participants were recruited to ingest herbal compositions and record the onset, duration, and intensity of cannabis-induced euphoria and/or dysphoria. Study participants took part in two separate tests: 1) use of a control substance, which included liquid cannabis extract dissolved in aqueous ethanol, and 2) use of a test substance, which included the liquid cannabis extract dissolved in aqueous ethanol, as well as SNAC.

Formulations. The selected cannabis concentrate is commercially available and was provided to participants in an ethanol solution. The concentrate contains 8 mg THC per dose. It was selected because it contains a high percentage of THC, which provides a noticeable effect on user-reported “euphoria”. Aqueous ethanol was used as solvent because it effectively dissolves cannabis extract, as well as SNAC.

Methods. For the Control experiment, each participant mixed the cannabis concentrate with 15 ml (one tablespoon) of aqueous ethanol, and immediately swallowed the mixture.

For the Test experiment, each participant mixed the cannabis concentrate with a pre-mixed solution of aqueous ethanol and 100 mg SNAC, and immediately swallowed the dissolved mixture.

For both the Control experiment and the Test experiment, each participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-10. Table 1 shows descriptions of euphoria and dysphoria levels for each scale value.

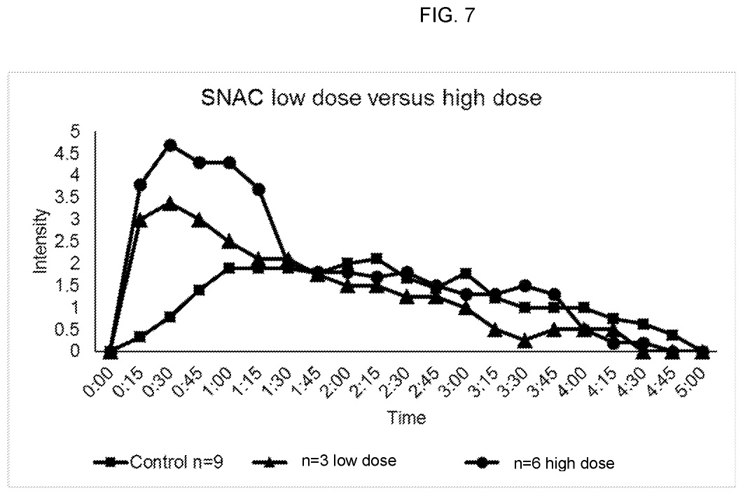

Results. The results are combined with the data from Example 2 and are reported for all participants in FIG. 7.

Onset: All three participants reported euphoria within five minutes of ingesting the cannabis/SNAC formulation (Test), with the time of onset ranging between two and five minutes. In contrast, the first time-point of euphoria reported by participants after ingestion of the cannabis-only formulation (Control) was fifteen minutes post-ingestion, with the time of onset ranging between fifteen minutes and one hour, fifteen minutes. By fifteen minutes post-ingestion, the average reported euphoria scale value was 3.0 for the cannabis/SNAC formulation (Test). In contrast, fifteen minutes after ingestion of the cannabis-only formulation (Control), the average reported euphoria scale value was 0.25.

Intensity: The average peak euphoria scale value after ingestion of the cannabis/SNAC formulation (Test) was 3.4, which occurred thirty minutes post-ingestion. In contrast, the highest average euphoria scale value after ingestion of the cannabis-only formulation (Control) was 2.2, which was at the two hour, fifteen minute time-point. Compared to Example 2 where the SNAC dose was 200 mg, the participants in Example 3 ingested only 100 mg of SNAC combined with the same quantity of cannabis used in Example 2. This reduced quantity of SNAC resulted in a reduced cannabis effect demonstrating a clear dose-response relationship between observed cannabis effect (euphoria) and SNAC dose. Consistent with Example 2, ingestion of the cannabis/SNAC formulation led to a higher peak intensity of euphoria, which occurred an average of one hour and forty-five minutes faster than when the cannabis-only formulation was ingested.

Duration: The results indicate that the addition of an absorption enhancer does not shorten the action of cannabis.

### Example 4

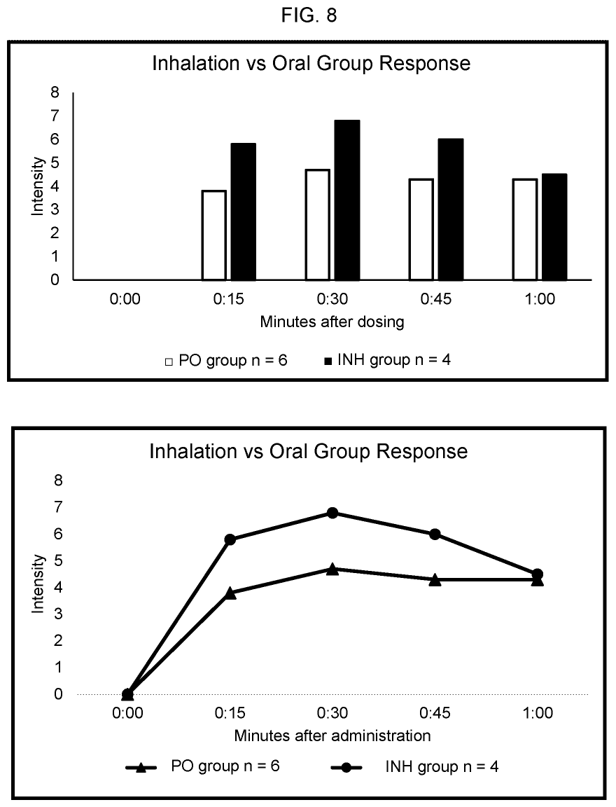

Inhalation versus oral group response (FIG. 8). Comparison of the pharmacodynamic response to inhaled and orally ingested herbal compositions of, in this example, cannabis, measured as subject-reported euphoria. Both the oral and inhaled groups reported similar time to peak effect (15-30 minutes). This is very surprising because oral cannabis is traditionally characterized by a very slow time to peak effect (up to 4 hours).

### Example 5

Summary of an herbal composition/SNAC oral rat pharmacokinetic (PK) study. The study was designed to characterize the pharmacokinetic profile of an herbal composition, cannabis extract, containing 56% THC/CBD in a 1:1 ratio (by weight) with and without the excipient, SNAC, following a single oral gavage administration to rats. In this study two doses of cannabis and SNAC and two ratios of cannabis to SNAC were tested. The experimental design is presented in Table 4 below.

TABLE 4
Experimental Design

| | | Extract¹ | SNAC | | | | |
|---|---|---|---|---|---|---|---|
| | | Dose | Dose | Dose | Dose | Dose | Number |
| Group | Group | Level | Level | Volume | Concentration² | Concentration³ | of |
| Nos | Designation | (mg/kg) | (mg/kg) | (mL/kg) | (mg API/mL) | (mg SNAC/mL) | Males |
| 1 | Excipient | 0 | 500 | 2 | 0 | 250 | 6 |
| | Control | | | | | | |
| 2 | Cannabis | 25 | 0 | 2 | 12.5 | 0 | 6 |
| | Control | | | | | | |
| 3 | Low Dose | 25 | 250 | 2 | 12.5 | 125 | 6 |
| 4 | Mid Dose | 25 | 500 | 2 | 12.5 | 250 | 6 |
| 5 | High Dose | 50 | 500 | 2 | 25 | 250 | 6 |

¹Extract contains 54% by weight (27% THC + 27% CBD) as the API (Active Pharmaceutical Ingredient)
²Dose of cannabis extract contains a mixture of THC:CBD in a ratio of 1:1 by weight
³SNAC dose is 10 times (THC + CBD) dose for groups 3 and 5 and 20 times for group 4.

Methods. Animals were dosed on Day 1 and a series of blood samples were collected over a period of 4 hours post dose for pharmacokinetic evaluation. Animals were euthanized following collection of their last blood sample.

Results. Following a single oral administration of an herbal composition containing THC/CBD in a 1:1 ratio combined with the absorption enhancing excipient (SNAC) at 25 mg extract/kg and 250 mg SNAC/kg (Group 3), 25 mg extract/kg and 500 mg SNAC/kg (Group 4), or 50 mg extract/kg and 500 mg SNAC/kg (Group 5), mean maximum concentration Cₘₐₓ ranged from 31.7 to 159.3 ng/mL for CBD and from 111.5 to 546.17 ng/mL for THC. The time to reach the mean maximum plasma concentration (Tₘₐₓ) ranged from 0.25 to 1 hour post dose for CBD and was reached at 1 hour post dose for the low and mid dose groups and at 2 hours post dose for the high dose group for THC. The AUC_{0-Tlast} ranged from 13.17 to 382.14 hr*ng/mL for CBD and from 170.64 to 1256.49 hr*ng/mL for THC.

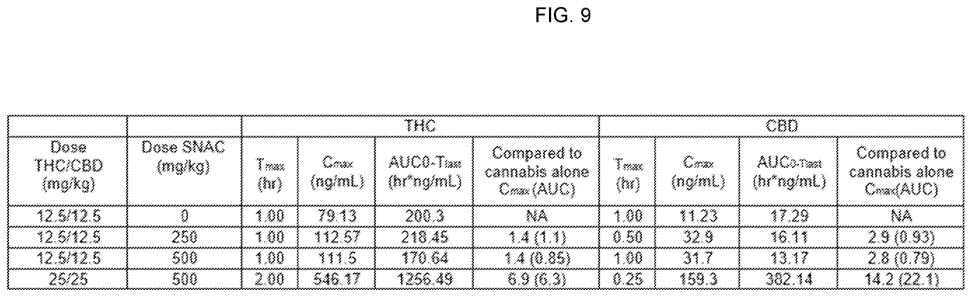

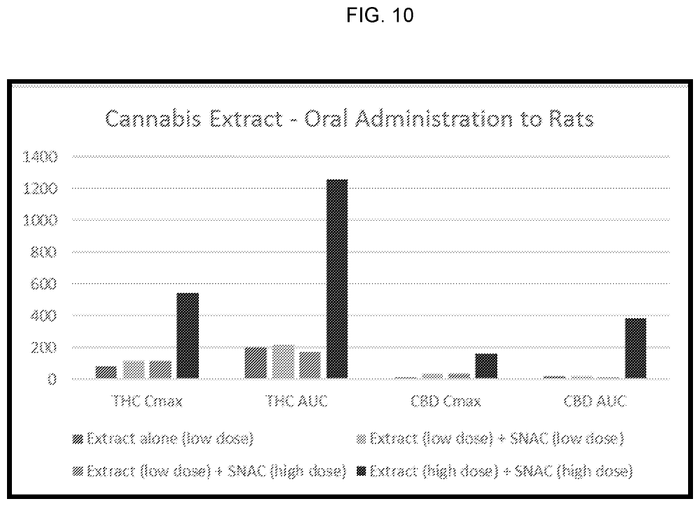

Over the dose range tested, Cₘₐₓ and AUC_{0-Tlast}, for THC was higher than for CBD. When administering the same cannabis extract (THC/CBD) dose (25 mg/kg total cannabinoid dose; 12.5 mg/kg THC/12.5 mg/kg CBD) with and without SNAC, for THC, a 1.4-fold Cₘₐₓ increase over cannabis alone was observed at SNAC doses of either 250 or 500 mg/kg. AUC was 1.1-fold greater in the 250 mg/kg SNAC group, but lower in the 500 mg/kg SNAC group, compared to the cannabis alone group. For CBD, 2.9-fold and 2.8-fold Cₘₐₓ increases over cannabis alone were observed at SNAC doses of either 250 or 500 mg/kg. AUC was lower in both groups, compared to the cannabis alone group. Increasing both the cannabis and SNAC doses 2-fold to 500 mg/kg SNAC and 50 mg/kg cannabis extract (25 mg/kg THC/25 mg/kg CBD), resulted in a 14.2-fold increase in the CBD Cₘₐₓ and a 6.9-fold increase in the THC Cₘₐₓ. AUC_{0-Tlast} for CBD and THC, were increased 22.1-fold and 6.3-fold, respectively (Table 5 and FIG. 9).

In summary, adding an absorption enhancer, such as SNAC, in an oral dosage formulation of an herbal composition provides faster onset of action and higher intensity of action at peak activity level of the herbal composition. These results indicate that SNAC improves the bioavailability of herbal compositions. Moreover, the absorption enhancer has no effect on the duration of action. The varying quantity of SNAC produces a clear dose-response relationship between observed effect of the herbal composition and SNAC dose.

### Example 6

Onset and duration of action of orally administered cannabis/NAC composition. This study was designed to assess the utility of the acid form of SNAC, N-[8-(2-hydroxybenzoyl) amino] caprylic acid (NAC), in enabling a rapid-acting oral form of cannabis.

Study Participant. One study participant was recruited to ingest cannabis compositions and record the onset, duration, and intensity of cannabis-induced euphoria and/or dysphoria. The study participant took part in two separate tests: 1) use of a control substance, which included cannabis concentrate oil in an herbal extract blend dissolved in aqueous ethanol, and 2) use of a test substance, which included the cannabis concentrate oil in an herbal extract blend dissolved in aqueous ethanol, as well as NAC.

Formulations. The selected cannabis concentrate oil is commercially available in a capsule and the contents of the capsule were provided to the participant in an ethanol solution. One capsule contains 9 mg CBD, 7.7 mg THC, herbal extract blend (Magnolia bark, Ashwagandha, Astragalus), and stearic acid (from vegetable oil), and the stated potency per capsule is: CBD 9.0 mg, THCA 0.0 mg and THC 7.6 mg. The formulation was selected because it provides a noticeable effect on user-reported “euphoria”, and the CBD content should ameliorate dysphoric effects if Test 2 delivers a very high dose of cannabinoids.

Methods. For the Control experiment, the participant mixed the cannabis concentrate with 15 ml (one tablespoon) of aqueous ethanol, and immediately swallowed the mixture.

For the Test experiment, the participant mixed the cannabis concentrate with 5 ml pre-mixed solution of aqueous ethanol and 100 mg NAC, and immediately swallowed the dissolved mixture.

For both the Control experiment and the Test experiment, the participant recorded the time of dose administration, the time of onset of euphoria and/or dysphoria, and the observed level of euphoria and/or dysphoria in fifteen minute intervals for five hours following administration of the cannabis dose. Euphoria and dysphoria were reported using a scale value, in a range from 1-5. Table 5 shows descriptions of euphoria and dysphoria levels for each scale value.

TABLE 5
Scale Values for Reporting Euphoria and Dysphoria

| Scale Value | Description |
|---|---|
| 0 | No observed effect |
| 1 | Mild observed effect; possibly psychological |
| 2 | Definite but mild effect |
| 3 | Definite substantial effect |
| 4 | Strong effect |
| 5 | Intense effect |

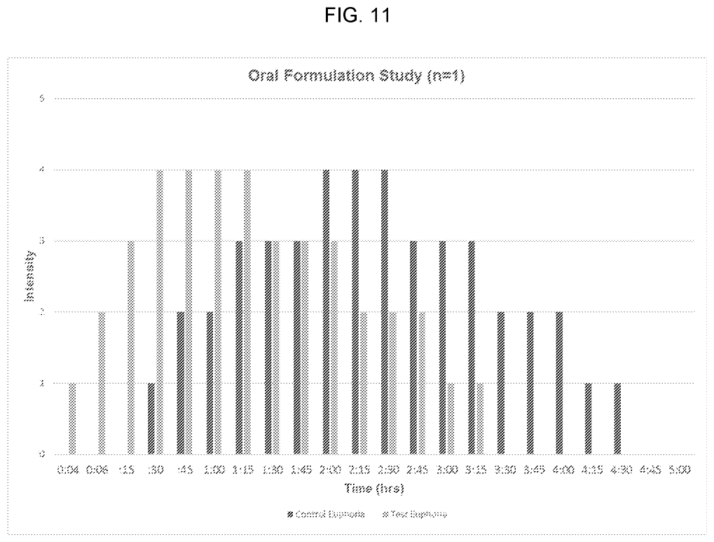

Results. The results shown below are scale values obtained for the participant in the control experiment (Table 6) and in the test experiment (Table 7). The values are plotted in FIG. 11.

TABLE 6
Control Experiment (n = 1)

| | Time from | Observed | Observed |
|---|---|---|---|
| Actual Time | Start | “Euphoria” | “Dysphoria” |
| 11:13 AM | 0:00 | (0-5) | (0-5) |
| 11:28 AM | :15 | 0 | 0 |
| 11:43 AM | :30 | 1 | 0 |
| 11:58 AM | :45 | 2 | 0 |
| 12:13 PM | 1:00 | 2 | 0 |
| 12:28 PM | 1:15 | 3 | 1 |
| 12:43 PM | 1:30 | 3 | 1 |
| 12:58 PM | 1:45 | 3 | 1 |
| 1:13 PM | 2:00 | 4 | 1 |
| 1:28 PM | 2:15 | 4 | 1 |
| 1:43 PM | 2:30 | 4 | 1 |
| 1:58 PM | 2:45 | 3 | 0 |
| 2:13 PM | 3:00 | 3 | 0 |
| 2:28 PM | 3:15 | 3 | 0 |
| 2:43 PM | 3:30 | 2 | 0 |
| 2:58 PM | 3:45 | 2 | 0 |
| 3:13 PM | 4:00 | 2 | 0 |
| 3:28 PM | 4:15 | 1 | 0 |
| 3:43 PM | 4:30 | 1 | 0 |
| 3:58 PM | 4:45 | 0 | 0 |
| 4:13 PM | 5:00 | 0 | 0 |

TABLE 7
Test Experiment (n = 1)

| | Time from | Observed | Observed |
|---|---|---|---|
| Actual Time | Start | “Euphoria” | “Dysphoria” |
| 11:20 AM | 0:00 | (0-5) | (0-5) |
| 11:24 AM | :04 | 1 | 0 |
| 11:26 AM | :06 | 2 | 0 |
| 11:35 AM | :15 | 3 | 1 |
| 11:50 AM | :30 | 4 | 0 |
| 12:05 PM | :45 | 4 | 0 |
| 12:20 PM | 1:00 | 4 | 0 |
| 12:35 PM | 1:15 | 4 | 0 |
| 12:50 PM | 1:30 | 3 | 0 |
| 1:05 PM | 1:45 | 3 | 0 |
| 1:20 PM | 2:00 | 3 | 0 |
| 1:35 PM | 2:15 | 2 | 0 |
| 1:50 PM | 2:30 | 2 | 0 |
| 2:05 PM | 2:45 | 2 | 0 |
| 2:20 PM | 3:00 | 1 | 0 |
| 2:35 PM | 3:15 | 1 | 0 |
| 2:50 PM | 3:30* | 0 | 0 |

*experiment ended

Onset: The participant reported euphoria within six minutes of ingesting the cannabis/NAC formulation (Test, Table 7 and FIG. 11). In contrast, the first time-point of euphoria reported by the participant after ingestion of the cannabis-only formulation (Control, Table 6 and FIG. 11) was forty-five minutes post-ingestion. By thirty minutes post-ingestion, the participant reported strong euphoria (scale value of 4) for the cannabis/NAC formulation. In contrast, thirty minutes after ingestion of the cannabis-only formulation, the participant only observed a mild effect that was possibly psychological (scale value of 1).

Intensity: The peak euphoria scale value after ingestion of both the cannabis-only (Control) and cannabis/NAC formulations (Test) was 4. However, peak intensity of euphoria was reached thirty minutes post-ingestion with the cannabis/NAC formulation (Test), whereas peak intensity of euphoria was reached two hours post-ingestion with the cannabis-only formulation (Control). Therefore, ingestion of the cannabis/NAC formulation led to a peak intensity of euphoria that occurred one hour and thirty minutes faster than when the cannabis-only formulation was ingested. The intensity of observed dysphoria was minimal for both the Test and Control, although the participant observed more mild dysphoria effects with the cannabis-only formulation (Control).

In summary, NAC, the acid form of SNAC, behaves similarly to SNAC when included in an oral dosage formulation of cannabis. A cannabis/NAC formulation provides faster onset of action as compared to a cannabis-only formulation.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in an administration benefit when assessed in an experimental protocol disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person ordinarily skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. An herbal composition with improved bioavailability formulated for oral delivery comprising (i) capsaicin, reserpine, vinblastine, hesperidin, naringin, rutin, quercitrin, curcumin, hypericin, resveratrol, catechin eugenol, limonene, or linalool and (ii) N-[8-(2-hydroxybenzoyl) amino] caprylate.

2. An herbal composition with improved bioavailability formulated for oral delivery comprising (i) vegetable matter derived from Curcuma longa, Hypericum perforatum, Polygonum cuspidatum, Vitis spp., Camellia sinsensis, Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., or Mentha spp. and (ii) an N-acylated fatty amino acid or salt thereof.

3. An herbal composition with improved bioavailability formulated for oral delivery comprising (i) an active component of Curcuma longa, Hypericum perforatum, Polygonum cuspidatum, Vitis spp., Camellia sinsensis, Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., or Mentha spp., wherein the active component has an aqueous solubility of less than 0.1 mg/ml and (ii) an N-acylated fatty amino acid or salt thereof.

4. An herbal composition with improved bioavailability formulated for oral delivery comprising (i) an extract of Curcuma longa, Hypericum perforatum, Polygonum cuspidatum, Vitis spp., Camellia sinsensis, Theobroma cacao, Capsicum spp., Rauwolfia vomitoria, Rauwolfia serpentina, Vinca spp., Citrus spp., Rheum rhabarbarum, Fagopyrum tataricum, Syzygium aromaticum, Lavandula spp., or Mentha spp., and (ii) an N-acylated fatty amino acid or salt thereof.

5. The herbal composition of claim 4 further comprising vegetable matter derived from Calophyllum brasiliense, Calophyllum caledonicurn, Calophyllum inophyllum, Calophyllum soulattri, Uncaria tomentosa, Thymus vulgaris, Matricaria recutita, Salix alba, Calendula officinalis, Usnea barbata, Ligusticum porterii-osha, Gaultheria procumbens, Camellia sinensis, Vaccinium myrtillus, Melissa officinalis, Allium sativum, Camellia sinensis, Krameria triandra, Punica granatum, Viburnum plicatum, Duboisia hopwoodii, Asclepias syriaca, Cannabis sativa, Cannabis indica, Cannabis ruderalis and/or Acer spp, or an extract thereof.

6. The herbal composition of claim 4 comprising capsaicin, reserpine, vinblastine, hesperidin, naringin, rutin, quercitrin, curcumin, hypericin, resveratrol, catechin eugenol, limonene, or linalool.

7. The herbal composition of claim 4, wherein the N-acylated fatty amino acid comprises one or more of Compounds I-XXXV (FIG. 3), or Compounds a-r (FIG. 4).

8. The herbal composition of claim 4, wherein the N-acylated fatty amino acid comprises monosodium-N-salicyloyl-8-aminocaprylate, disodium-N-salicyloyl-8-aminocaprylate, or N-(salicyloyl)-8-aminocaprylic acid.

9. The herbal composition of claim 4, wherein the N-acylated fatty amino acid or a salt thereof comprises 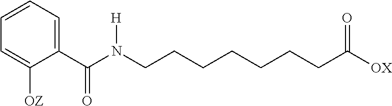
wherein X and Z are independently H, a monovalent cation, a divalent metal cation, or an organic cation.

10. The herbal composition of claim 9, wherein the monovalent cation is sodium or potassium.

11. The herbal composition of claim 9, wherein the divalent metal cation is calcium or magnesium.

12. The herbal composition of claim 9, wherein the organic cation is ammonium or tetramethylammonium.

13. The herbal composition of claim 9, wherein X is H.

14. The herbal composition of claim 9, wherein X is a monovalent cation comprising sodium or potassium.

15. The herbal composition of claim 9, wherein X is a divalent metal cation comprising calcium or magnesium.

16. The herbal composition of claim 9, wherein X is an organic cation comprising ammonium or tetramethylammonium.

17. The herbal composition of claim 9, wherein Z is H.

18. The herbal composition of claim 9, wherein Z is a monovalent cation comprising sodium or potassium.

19. The herbal composition of claim 9, wherein Z is a divalent cation comprising calcium or magnesium.

20. The herbal composition of claim 9, wherein X is H and Z is H.

21. The herbal composition of claim 9, wherein X is H and Z is sodium.

22. The herbal composition of claim 9, wherein X is sodium and Z is sodium.

23. The herbal composition of claim 4 wherein the N-acylated fatty amino acid or salt thereof provides an administration benefit.

24. The herbal composition of claim 23 wherein the administration benefit is a dose-dependent administration benefit.

25. The herbal composition of claim 24 wherein the dose-dependent administration benefit is at a dose of 100-200 mg.

26. The herbal composition of claim 23 wherein the administration benefit comprises one or more of increased absorption of a measured component of vegetable matter, increased bioavailability of a measured component of vegetable matter, faster onset of action of a measured component of vegetable matter, higher peak concentrations of a measured component of vegetable matter, faster time to peak concentrations of a measured component of vegetable matter, increased subjective therapeutic efficacy, increased objective therapeutic efficacy, improved taste, and improved mouthfeel as compared to a control composition without the N-acylated fatty amino acid.

27. The herbal composition of claim 4 comprising a botanical medicine.

28. The herbal composition of claim 4 comprising a nutritional supplement.

29. The herbal composition of claim 4 comprising a botanical product.

30. The herbal composition of claim 4 comprising a surfactant, detergent, azone, pyrrolidone, glycol or bile salt.

31. The herbal composition of claim 4 comprising a therapeutically effective amount of the vegetable matter.

32. The herbal composition of claim 31 wherein the therapeutically effective amount treats a symptom of acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, and withdrawal.

33. The herbal composition of claim 4 comprising vitamins or minerals.

34. The herbal composition of claim 4 comprising vitamins and minerals.

35. The herbal composition of claim 33 wherein the vitamins are selected from one or more of Vitamin A, Vitamin B1, Vitamin B6, Vitamin B12, Vitamin C, Vitamin D, Vitamin E, or Vitamin K.

36. The herbal composition of claim 33 wherein the minerals are selected from one or more of calcium, chromium, iodine, iron, magnesium, selenium and/or zinc.

37. An oral formulation comprising an herbal composition of claim 1-36.

38. An oral formulation of claim 37 wherein the oral formulation is swallowable or chewable.

39. An oral formulation of claim 37 wherein the oral formulation is liquid or solid.

40. An oral formulation of claim 37 wherein the oral formulation is a solution, suspension, or spray.

41. An oral formulation of claim 37 wherein the oral formulation is a tablet, capsule or sachet.

42. An oral formulation of claim 37 wherein the oral formulation is flavored.

43. A method of preparing an oral formulation of an herbal composition having improved bioavailability, wherein the method comprises adding an absorption enhancer to the oral formulation of the herbal composition and wherein the oral formulation of the herbal composition has improved bioavailability compared to an oral formulation of the herbal composition without an absorption enhancer.

44. The method of claim 43, wherein the absorption enhancer is an N-acylated fatty amino acid or a salt thereof.

45. The method of claim 44, wherein the N-acylated fatty amino acid or a salt thereof comprises 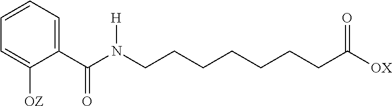
wherein X and Z are independently H, a monovalent cation, a divalent metal cation, or an organic cation.

46. The method of claim 44, wherein the N-acylated fatty amino acid is selected from monosodium-N-salicyloyl-8-aminocaprylate, disodium-N-salicyloyl-8-aminocaprylate, and N-(salicyloyl)-8-aminocaprylic acid.

47. A method of treating a subject in need thereof comprising administering a therapeutically effective amount of a composition of claim 4 to the subject thereby treating the subject in need thereof.

48. A method of claim 47 wherein the therapeutically effective amount provides an effective amount, a prophylactic treatment, and/or a therapeutic treatment.

49. A method of reducing or eliminating one or more symptoms of a disease or disorder in a human subject,
wherein said method comprises delivering a therapeutically effective amount of a composition of claim 4 to the subject, thereby reducing or eliminating one or more symptoms of the disease or disorder, and
wherein said disease or disorder is acquired hypothyroidism, acute gastritis, addiction, ADHD, agoraphobia, AIDS, AIDS-related anorexia, alcoholism, Alzheimer's disease, amyotrophic lateral sclerosis (ALS), ankyloses, anxiety, arthritis, Asperger's syndrome, asthma, atherosclerosis, autism, auto-immune diseases, bacterial infections, bipolar disorder, bone loss, blood disorders, brain injury/stroke, cachexia, cancer, carpal tunnel syndrome, cerebral palsy, cervical disk disease, cervicobrachial syndrome, chronic fatigue syndrome, chronic pain, cluster headache, conjunctivitis, Crohn's disease, cystic fibrosis, depression, dermatitis, diabetes, dystonia, eating disorders, eczema, epilepsy, fever, fibromyalgia, flu, fungal infection, gastrointestinal disorders, glaucoma, glioma, Grave's disease, heart disease hepatitis, herpes, Huntington's disease, hypertension, impotence, incontinence, infant mortality, inflammation, inflammatory bowel disease (IBD), insomnia, liver fibrosis, mad cow disease, menopause, metabolic disorders, migraine headaches, motion sickness, MRSA, multiple sclerosis (MS), muscular dystrophy, mucosal lesions, nail patella syndrome, nausea and vomiting associated with cancer chemotherapy, neuroinflammation, nicotine addiction, obesity, obsessive compulsive disorder (OCD), osteoporosis, osteopenia, pain, pancreatitis, panic disorder, Parkinson's disease, periodontal disease, peripheral neuropathy, phantom limb pain, poison ivy allergy, premenstrual syndrome (PMS), proximal myotonic myopathy, post-traumatic stress disorder (PTSD), psoriasis, Raynaud's disease, restless leg syndrome, schizophrenia, scleroderma, septic shock, shingles herpes zoster), sickle cell disease, seizures, sleep apnea, sleep disorders, spinal injuries, stress, stuttering, temporomandibular joint disorder (TMJ), tension headaches, tinnitus, Tourette's syndrome, traumatic memories, wasting syndrome, or withdrawal syndrome.
